(19) United States
(12) Patent Application Publication
(10) Pub. No.: US 2019/0117698 A1
(43) Pub. Date: Apr. 25, 2019

(54) METHODS AND COMPOSITIONS FOR TREATING VASCULAR-RELATED DEGENERATIVE NEUROLOGICAL DISORDERS

(71) Applicant: HENRY DOUGLAS BROESKA, LAGUNA WOODS, CA (US)

(72) Inventor: HENRY DOUGLAS BROESKA, LAGUNA WOODS, CA (US)

(21) Appl. No.: 15/793,079

(22) Filed: Oct. 25, 2017

(51) Int. Cl. A61K 35/28; A61P 25/28; A61K 9/00; A61M 25/10

(52) U.S. Cl. CPC A61K 35/28; A61P 25/28; A61K 2035/124; A61M 25/1002; A61K 9/0019

(57) ABSTRACT

Methods and compositions for treating a patient having a vascular-related degenerative neurological disorder are disclosed. The methods comprising the steps of administering autologous bone marrow stem cells into an internal jugular vein of the patient and subsequently occluding the vein so as to create a local retrograde flow of the stem cells in the cranial veins of the patient. The further step of administering the autologous bone marrow stem cells to the patient's spinal canal subsequent to the administration of the cells to the jugular vein may also be performed. Further disclosed are pharmaceutical compositions for use in such methods. The neurodegenerative disorder commonly treated by the present invention is multiple sclerosis.

### FIELD OF THE INVENTION

The invention relates to methods and compositions for treating a patient having or at risk of developing a vascular-related degenerative neurological disease(s) or disorder(s), e.g. multiple sclerosis, by administering stem cells to the patient.

### BACKGROUND OF THE INVENTION

Vascular-related degenerative diseases afflict many people and are often neurologically based. Vascular dysfunction with respect to craniovertebral anomalies offers an explanation for symptoms of some neurological diseases for which there are no effective treatments. In the case of neuronal disorders that have a primary vascularorigin, circulating neurotoxins cross the blood brain barrier (BBB) to reach neuronal targets and trigger injury (de Vries H et al, 1997, Zlokovic B. 2008, Banks W A et al 2013, Minangar A et al, 2003). Moreover, despite medications that are prescribed only for symptoms of the effect, these diseases are often fatal. One of the effects of vascular-related diseases results is the destruction of the myelin sheath covering neurons necessary for the proper function of the central nervous system (Steinman L, 2001). Multiple Sclerosis is one such vascular-related degenerative neurological disease having serious, sometimes life-threatening consequences. (Schelling F. 1986)

Medical evidence indicates that MS begins as a brain blood vessel disorder, which leads to progressive neurological impairment (Kirk J et al 2003, D'haeseleer M. 2011, Zlokovic B, 2008). There are an estimated 400,000 MS patients in the US alone with a worldwide population of 2.5 million. This prevalence is expected to increase greatly by the year 2025. Moreover, because patients experience progressive functional and physical decline, MS is very treatment intensive and as a consequence, very expensive to treat. In the future, MS will occupy a greater share of U.S. and world health care budgets (Adelman G et al 2013, Hartung D M, 2015). Most patients are diagnosed between the ages of 20 and 50, and about two thirds are women. The disease is more frequently found among people raised at higher latitudes. Decreased exposure to the sun resulting in decreased vitamin D production has been suggested as a possible factor (Alharbi F M. 2015). Studies also indicate that genetic factors make certain patients susceptible to the disease, but there is no evidence that MS is directly inherited. What is Multiple Sclerosis (n.d.) Retrieved from http://www.nationalmssociety.org/NationalMSSociety/media/MSNationalFiles/Brochures/Brochure-What-Is-MS.pdf.

MS is a neurodegenerative disease in which vascular abnormalities result in a cascade of events that culminates in white matter inflammation developing at locations in the brain adjacent to dysfunctional veins that have become porous and unable to contain blood proteins from crossing the blood brain barrier (Schelling F, 2014).

Inflammation subsequently causes destruction of the myelin sheath (the fatty substance that coats and protects nerve fibers in the brain and spinal cord) covering neurons and axonal fibers leading to a range of signs and symptoms including improper balance, lack of normal walking ability and impaired cognition. In magnetic resonance images (MRIs), the regions involved eventually appear as white areas, or scleroses within normal-appearing brain or spinal cord tissue. Currently, MS is considered among many in the medical community to be an autoimmune disease where the immune system attacks myelin idopathically, meaning for an unknown reason, or spontaneously. (Weinshenker B G, 1994).

The bold and time-worn assertion that MS is an autoimmune disease is not without challenge in 2016, despite its presence on nearly every list of such diseases. The main criterion of any given autoimmune disease is that a precise auto-antigen must be present in all patients with the disease. Despite decades of attempts to identify various proteins, lipids, and gangliosides in myelin as potential MS antigens, none have been found or confirmed, nor does MS meet any of the other five criteria necessary for that statement (Wootla B, 2012). For example, investigators have attempted to show that CD4+T cells play a pathogenic role in the development of MS. Unfortunately, many findings regarding the role of CD4+ T cells have not been reproduced elsewhere. Even a cursory review of the literature will reveal that potential explanations of the pathophysiology of MS abounds with confusion as researchers endeavor to classify the disease as either pathological or clinical depending on their predisposition. Instead, the diagnosis of MS does not rely on either sound pathognomonic or observational criteria, but a combination of ruling out other diseases and multiple other tests (Polman C. et al 2011).

Of course the immune system plays a critical role in the development of lesions, especially during the acute early phases of the disease characterized by inflammation, often leading to relapses. Recent studies at least partially refute the autoimmune hypothesis (Correale J et al 2006, Wootla B, 2012). However, the hypothesis described herein that explains the immune-mediated pathogenesis of MS is by no means newly-conceived; it is the oldest clinical evidence that exists as to the cause. For almost 200 years, physicians have observed that the autoimmune response in MS is secondary to the vascular disorder. Upon a review of the literature over generations, it is clear that a significant number of researchers, including Jean-Martin Charcot, the Father of Modern Neurology, have convincingly demonstrated the Vein-Lesion Connection in MS, and there is much experimental evidence to support the hypothesis (Ge Y 2008, Kumar D 2011, Gulcher J R, 1994, Chaudhuri A 2004, Schelling F 2012).

The disease is characterized in its first stages by a failure of or leaking of fluid through the blood brain barrier (BBB) and subsequent infiltration and local dispersal of blood-borne inflammatory cytokines into the central nervous system (CNS) (Krizanac-Bengez I et al 2004, Larochelle C et al 2011).

There are three main courses, or patterns of progression in multiple sclerosis (Poser C M. 1983). In each case, MS symptoms may be mild, moderate or severe. The first type of MS is Relapsing-Remitting MS (RRMS) in which there are acute and unpredictable exacerbations (acute attacks, also called flare-ups). Over 80% of patients start off with this type. During this period symptoms get worse and there are periods of full or partial recovery, but sometimes no recovery at all. The attacks may evolve over days or even weeks, and recovery can take weeks, or even months. In between the attacks there is calm, and symptoms do not worsen. Although there are over a dozen medications approved by the U.S. Food and Drug Administration (FDA) for the treatment of relapsing-remitting MS (RRMS), clinical evidence on whether any of them significantly impede or slow the disease progression is conflicting.

The second type is Primary-Progressive MS (PPMS). About 15% of patients have this type. There are no clear relapses or remissions and the progression of the disease is steady. It is the most common form of MS in those who develop the disease after 40 years of age. Currently there are no medications that have been specifically approved by the FDA for the treatment of primary-progressive MS. However, many neurologists prescribe the medications indicated for the relapsing-remitting course in the hope it may slow progression.

The third type is Secondary-Progressive MS (SPMS) which starts off as the relapsing-remitting type of multiple sclerosis. Relapses and partial recoveries occur. However, in between the cycles, ever-increasing disability does not go away. Eventually it becomes a progressive disease with no cycles. The progressive stage may start very early on, or years, and even decades later. With few risk-free alternatives available, neurologists often continue to prescribe the same medications that the patient took during the relapsing-remitting phase beyond the point where there is evidence that the drug is able to do an acceptable job of controlling disease activity (Lublin FD. et al. 2014).

Signs and symptoms of MS vary widely and depend on the amount of nerve damage and which nerves are affected. Two of the most common symptoms are fatigue and difficulty walking. About 80 percent of people with MS report having fatigue. Fatigue that occurs with MS is more than just feeling tired. It can become debilitating, affecting the ability to work, focus on and perform everyday tasks.

Common symptoms of multiple sclerosis are bladder problems, bowel problems, cognitive function, depression, emotional changes, dizziness and vertigo, numbness or weakness, sexual dysfunction, spasticity and muscle spasms, tremor, vision problems and gait changes. The most commonly reported cognitive abnormalities include executive functional challenges with memory, abstraction, attention and word finding. Nordqvist, C., Multiple Sclerosis: Causes, Symptoms and Treatments (2016, Mar. 9). Retrieved from http://www.medicalnewstoday.com/articles/37556.php?page=2.

It isn't clear why MS develops in some people and not others. A combination of genetics and environmental factors appears to be responsible although that does not explain entire ethnic groups who are at low risk within populations where the disease is prevalent. Many factors may increase the risk of developing multiple sclerosis. Age is a factor since, while MS can occur at any age, it most commonly affects people between the ages of 15 and 60, with many fewer cases seen before the age of 30. Retrieved from “How age affects Multiple Sclerosis Symptoms and Progression”. 2015 http://www.nationalmssociety.org/What-is-MS/Who-Gets-MS. Polliack M L et al 2001) Another factor is gender with women being about twice as likely as men to develop MS. Family history is also a factor since, if parents or siblings have MS, one is at a higher risk of developing the disease. Other factors include certain infections, for example, a variety of viruses have been linked to MS, including Epstein-Barr, the virus that causes infectious mononucleosis, herpes virus 6, herpes simplex virus, influenza, measles, mumps, varicella-zoster virus, cytomegalovirus (CMV), and respiratory syncytial virus (RSV) and Chlamydia pneumoniae, a bacterial infection. There is no evidence that any type of vaccine causes multiple sclerosis. Retrieved from Multiple Sclerosis 2012 http://umm.edu/health/medical/reports/articles/multiple-sclerosis. Caucasians, particularly those of Northern European descent, are at the highest risk of developing MS. People of Asian, African or Native American descent have the lowest risk. MS is far more common in countries of high latitudes, including Canada, the northern United States, the UK, Scotland, Scandinavia, New Zealand, southeastern Australia, northern Europe, Russia, and northern Asia (Simpson Jr. S et al 2011). Certain autoimmune diseases are a factor since a person has a slightly higher risk of developing MS if they have thyroid disease, type 1 diabetes or inflammatory bowel disease. Smokers who experience an initial event of symptoms that may signal MS are more likely than nonsmokers to develop a second event that confirms relapsing-remitting MS. Multiple Sclerosis (n.d.) Retrieved from http://www.mayoclinic.org/diseases-conditions/multiple-sclerosis/symptoms-causes/dxc-20131884. Individuals with a traumatic brain injury are also at an increased risk of developing MS. (Freire M A 2012).

A medical history and complete neurologic exam and some lab tests along with MRI imaging are needed to diagnose MS. There are no specific tests for MS. Instead, a diagnosis of multiple sclerosis often relies on ruling out other conditions that might produce similar signs and symptoms, known as a differential diagnosis. Doctors are likely to start with a thorough medical history and examination. Doctors may then recommend blood tests to help rule out other diseases with symptoms similar to MS. Tests to check for specific biomarkers associated with MS are currently being sought but the consensus revision of Diagnostic criteria for multiple sclerosis: 2010 Revisions to the McDonald criteria is the gold standard for diagnosing the disease. (Polman C. et al 2011)

Also recommended is a lumbar puncture (spinal tap), in which a small sample of cerebrospinal fluid (CSF) is removed from the spinal canal for laboratory analysis. This sample can show abnormalities in proteins, products of myelin destruction and antibodies that correlate closely with the clinical activity of MS (Cohen, S R 1980); specifically oligoclonal IgG bands (not found in the serum of the patient) or an elevated IgG index. A spinal tap can also help rule out infections and other conditions with symptoms similar to MS (Polman C. et al 2011).

An MRI may also be recommended, which can reveal areas of MS (lesions) on the brain and spinal cord. A patient may receive an intravenous injection of a contrast material to highlight lesions that indicate the disease is in an active phase. (Polman C, et al. 2011)

In most people with relapsing-remitting MS, the diagnosis is fairly straightforward and based on a pattern of symptoms consistent with the disease and confirmed by brain imaging scans, such as MRI.

Diagnosing MS can be more difficult in persons with unusual symptoms or a progressive early form of the disease. In these cases, further testing with spinal fluid analysis and additional imaging may be needed. Pietrangelo, A. Understanding Multiple Sclerosis. (n.d.) Retrieved from www.healthline.com/health/multiple-sclerosis, (Polman C, et al. 2011)

There is currently no cure for multiple sclerosis. Patients diagnosed with MS have been subject to a decline of functional abilities until death. MS patients live 10-15 years less than average despite available medications.

Interventional pharma-based therapies are targeted at symptomatic treatment only and have not been effective for either a reduction of symptoms over time or restoration of function. Historically, the standard treatment for Multiple Sclerosis (MS) and other chronic neurological diseases of a demyelinating nature has been mainly pharmacological, with the intent of returning neurological function after an attack, inhibiting new attacks and preventing disability. (Rice C. 2014)

Medications for MS have long-term side effects such as muscle aches, fatigue, cardiac issues, allergic reactions and headaches, and additionally some are immunosuppressive. Moreover, because of the degenerative nature of MS, the disease can only be minimally controlled, not cured. Therefore, as these drugs only modify symptoms and do not slow down the rate at which neurons are lost, it is necessary to increase dosages or introduce new medication, such as (beta) 3-inhibitors. These increased dosages by themselves introduce more potential health risks to patients.

Treatments for acute attacks or flares of MS may include oral or intravenous corticosteroids or plasma exchange. Corticosteroids, such as oral prednisone and intravenous methylprednisolone, are prescribed to reduce nerve inflammation. Side effects may include insomnia, increased blood pressure, mood swings, elevated blood sugar, drug-induced psychosis and fluid retention.

Plasma exchange electrophoresis is a technique where the liquid portion part of blood (plasma) is removed and separated from blood cells. The blood cells are then mixed with a protein solution (albumin) and put back into the body. Plasma exchange may be used if symptoms are new, severe and haven't responded to steroids (Tumani H. 2008).

At this time, there are no FDA-approved treatments available for slowing the progression of primary-progressive MS (PPMS) or secondary-progressive MS (SPMS). For relapsing-remitting MS (RRMS), several disease-modifying therapies are available. Much of the immune response associated with MS occurs in the early stages of the disease. It is claimed that aggressive treatment with these medications as early as possible can lower the relapse rate and slow the formation of new lesions. Many of the disease-modifying therapies used to treat MS carry significant health risks. Selecting the right therapy depends on careful consideration of many factors, including duration and severity of disease, effectiveness of previous MS treatments, other health issues, cost, and childbearing status.

Oral pharmacologic treatments are numerous and include beta interferons which are among the most commonly prescribed medications for the treatment of MS. They are injected under the skin or into muscle and can reduce the frequency and severity of relapses, although there is no evidence they change the course or timelines of the disease. Glatiramer acetate (Copaxone ®) may help block the immune system's attack on myelin and must be injected beneath the skin. Teriflunomide (Aubagio®) is a once-daily medication which may reduce relapse rate. Natalizumab (Tysabri ®) is designed to block the movement of potentially damaging immune cells from the bloodstream to the brain and spinal cord. It has been used as a first line treatment for some people with severe MS or as a second line treatment in others. Dimethyl fumarate, (Tecfidera®) has been approved for the treatment of patients with relapsing forms of multiple sclerosis. Alemtuzumab, (Lemtrada®) has been approved for the reduction of relapses of MS by targeting a protein on the surface of immune cells and depleting white blood cells. This effect is claimed by the manufacturer to potentially limit nerve damage caused by the white blood cells, but it also increases the risk of infections and autoimmune disorders. (Rice C. 2014)

In some cases, treatments have been provided for MS signs and symptoms such as physical therapy and even possibly muscle relaxants such as baclofen (Lioresal ®) and tizanidine (Zanaflex®).

In other cases medications have been administered to reduce fatigue. Further, medications have been prescribed for depression, pain, sexual dysfunction, and bladderor bowel control problems that are associated with MS.

Activities such as exercise, meditation, yoga, massage, eating a healthier diet, acupuncture and relaxation techniques may help boost overall mental and physical well-being, but there are few studies to back up their use in managing symptoms of MS. Multiple Sclerosis (n.d.). Retrieved from http://www.mayoclinic.org/diseases-conditions/multiple-sclerosis/diagnosis-treatment/treatment/txc-20131903.

Other potential treatments include minimally invasive dilatation of the jugular veins, put forward in a hypothesis by Paolo Zamboni in 2009. Clinical trials have not proven the efficacy of vein dilatation because of the jugular veins' tendency to restenose after a short time (average <90 days). Furthermore, much anecdotal evidence of damage intraluminally has been reported by MS patients as a result of neck vein catheterization and dilatation. Accordingly, the problems with this technique render this process ineffective. (Tsivgoulis G, 2015.)

Stem cell therapy for the treatment of MS has been attempted wherein most or all clinical human studies using stem cells for the treatment of MS employ the administration of a particular amount of stem cells into the hand or the arm of the patient. The dorsal metacarpal veins in the hand and the cephalic and basilic veins in the forearm are the veins most often referred to or used for initiating intravenous (IV) therapy when infusing stem cells. The methodologic problem with using such a peripheral vein, including the IJV to infuse stem cells is that many of the infused stem cells will become sequestered in the lungs and to a lesser extent the liver and spleen and not reach the brain of the MS patient (Fischer, U M, 2009, Furlani et al 2009).

Stem cell therapy has further been used in attempts to treat other diseases and conditions, for example, they have been used to treat myocardial and spinal injury as disclosed in US Application 2005/0015048, US application 2013/0338637 and US application 2012/0156230.

Despite all such advances and attempts, a need remains for a more effective treatment of a patient suffering from vascular-related degenerative neurological disease(s), particularly multiple sclerosis. The current invention is directed to this and other needs.

### SUMMARY OF THE INVENTION

In one aspect the present invention provides a method of treating a patient having a vascular-related degenerative neurological disease comprising intravenously administering to the patient a therapeutically effective amount of stem cells to the patient.

Another aspect the present invention provides a method of treating a patient having a vascular-related degenerative neurological disease wherein the vascular-related degenerative neurological disease is selected from the group consisting of optic neuritis, Devic's disease, Lyme's disease, migraine headache, cluster headache, vascular dementia, ischemic depression, ischemic stroke, motor neuron disease, multiple sclerosis and other acquired myelinoclastic disorders.

In yet another aspect the present invention provides a method of treating a patient having multiple sclerosis comprising intravenously administering to the patient a therapeutically effective amount of stem cells.

Another aspect the present invention provides a method of treating a patient having multiple sclerosis comprising intravenously administering to the patient a therapeutically effective amount of stem cells wherein the intravenous administration is to a vein of the patient wherein the vein is selected from the group consisting of the internal jugular veins, azygous veins and vertebral veins.

Yet another aspect the present invention provides a method of treating a patient having multiple sclerosis comprising intravenously administering to the patient a therapeutically effective amount of stem cells wherein the intravenous administration is to an internal jugular vein of the patient.

In still another aspect of the present invention there is provided a method of treating a patient having multiple sclerosis comprising intravenously administering to the patient a therapeutically effective amount of autologous bone marrow stem cells to the patient.

In yet another aspect of the present invention there is provided a method of treating a patient having multiple sclerosis comprising intravenously administering to the patient a therapeutically effective amount of autologous bone marrow stem cells wherein the intravenous administration is to an internal jugular vein of the patient whereby the stem cells migrate to the cranial veins of the brain of the patient.

In still another aspect of the present invention there is provided a method of treating a patient having multiple sclerosis comprising administering to the internal jugular vein of the patient a therapeutically effective amount of autologous bone marrow stem cells whereby the stem cells migrate to the cranial veins of the patient and are maintained in the cranial veins by expansion of a device wherein the expansion of the device occludes the vein proximal to the point of release of the stem cells.

In another aspect of the present invention there is provided a method of treating a patient having multiple sclerosis comprising administering to the internal jugular vein of the patient a therapeutically effective amount of autologous bone marrow stem cells whereby the stem cells migrate to the cranial veins of the patient and are maintained in the cranial veins by expansion of a device wherein the expansion of the device occludes the vein proximal to the point of release of the stem cells and a local retrograde flow of the cells is thereby maintained in the cranial veins.

In still another aspect of the present invention there is provided a method of treating a patient having multiple sclerosis comprising administering to the internal jugular vein of the patient a therapeutically effective amount of autologous bone marrow stem cells whereby the stem cells migrate to the cranial veins of the patient and are maintained in the cranial veins by expansion of a balloon tipped catheter having the cells releasably associated therewith wherein the expansion of the device occludes the vein proximal to the point of release of the stem cells and a local retrograde flow of the cells is thereby maintained in the cranial veins.

In yet another aspect of the present invention there is provided a method of treating a patient having multiple sclerosis comprising intravenously administering to the patient a therapeutically effective amount of autologous bone marrow stem cells to the patient wherein the therapeutically effective amount of the autologous bone marrowstem cells comprises between about 300 million and about 1 billion autologous bone marrow stem cells isolated from the patient.

In another aspect of the present invention the autologous bone marrow stem cells are intravenously administered to an internal jugular vein of the patient for a period of time not less than about 30 seconds to about not more than about 60 seconds at any one time before being repeated if necessary.

In still another aspect of the present invention there is provided a pharmaceutical composition comprising between about 300 million and about 500 million autologous bone marrow stem cells and a pharmaceutically acceptable medium.

In yet another aspect of the present invention there is provided a pharmaceutical composition comprising between about 300 million and about 500 million autologous bone marrow stem cells and a pharmaceutically acceptable medium wherein the pharmaceutically acceptable medium comprises normal saline.

In another aspect of the present invention there is provided a pharmaceutical medium comprising Dulbecco's-modified Eagle's Medium-Low Glucose, GlutaMAX™, penicillin, streptomycin and fetal calf serum.

In another aspect of the present invention there is provided a method of treating a patient having a vascular-related degenerative neurological disease, in particular multiple sclerosis, comprising the steps of:
selectively transfusing a therapeutically effective amount of autologous bone marrow stem cells into an internal jugular vein of the patient; and
occluding the jugular vein proximally to the point of insertion of the cells through expansion of a balloon tipped catheter thereby maintaining a local retrograde flow of the cells in the cranial vein.

In another aspect of the present invention there is provided a method of treating a vascular-related degenerative neurological disease, in particular multiple sclerosis, comprising the further step of intrathecally administering a therapeutically effective amount of autologous bone marrow stem cells to the patient's spinal canal subsequent to the administration of a therapeutically effective amount of autologous bone marrow stem cells to an internal jugular vein of the patient.

In another aspect the present invention provides a method of treating a patient having a vascular-related degenerative neurological disease comprising intravenously administering a therapeutically effective amount of stem cells to the patient; and thereafter intrathecally administering a therapeutically effective amount of stem cells to the patient.

In still another aspect the present invention provides a method of treating multiple sclerosis comprising intravenously administering a therapeutically effective amount of stem cells to the patient; and thereafter intrathecally administering a therapeutically effective amount of stem cells to the patient.

In yet another aspect the present invention provides a method of treating multiple sclerosis comprising intravenously administering a therapeutically effective amount of stem cells to the patient; and thereafter intrathecally administering a therapeutically effective amount of stem cells to the patient wherein the intravenous administration is to an internal jugular vein of the patient and intrathecal administration is to the patient's spinal canal.

In another aspect the present invention provides a method of treating multiple sclerosis comprising intravenously administering to an internal jugular vein of a patient a therapeutically effective amount of stem cells to the patient; and thereafter administering to the patient's spinal canal a therapeutically effective amount of stem cells wherein the intravenous administration is by a balloon tipped catheter which expands proximally to the cells to occlude the jugular vein such that the cells are maintained in the cranial veins and undergo a local retrograde flow and the intrathecal administration is to the patient's spinal canal by lumbar puncture whereby the intrathecal administration is performed about 4 days subsequent to the intravenous administration.

In another aspect the present invention provides a method of preparing a pharmaceutical composition comprising a therapeutically effective amount of autologous stem cells and a pharmaceutically acceptable carrier comprising removing the bone marrow from the iliac crest of the patient to be treated; isolating the cells by centrifugation and mixing the cells with EDTA and anticoagulant in a serum gel tube (Sodium Heparin tube).

In still another aspect the present invention provides a device comprising autologous bone marrow stem cells releasably associated therewith.

In yet another aspect the present invention provides an expandable balloon tipped catheter comprising autologous bone marrow stem cells releasably associated therewith.

For the purpose of promoting an understanding of the principles of the invention, reference will now be made to the embodiments of the invention and specific languagewill be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

### DETAILED DESCRIPTION OF THE INVENTION

Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use the invention.

As used herein, the terms “effective,” “effective amount,” and “therapeutically effective amount” refer to that amount of stem cells and/or a pharmaceutical composition thereof that results in amelioration of symptoms, a slowing of the progression of ora prolongation of survival in a subject with a vascular-related degenerative disorder, e.g. multiple sclerosis. A therapeutically relevant effect relieves to some extent one or more symptoms of a vascular-related degenerative disorder, e.g. multiple sclerosis, or returns to normal either partially or completely one or more physiological or biochemical parameters associated with or causative of a vascular-related degenerative disorder, e.g. multiple sclerosis.

As used herein, the terms “treat,” “treatment,” or “treating,” refer to therapeutic treatments, wherein the object is to reverse, alleviate, ameliorate, inhibit, prevent, slow down or stop the progression or severity of a condition associated with a disease or disorder. The term “treating” includes reducing or alleviating at least one adverse effect or symptom of a condition, disease or disorder. Treatment is generally effective if one or more symptoms or clinical markers are reduced. Alternatively, treatment is effective if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptoms, diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not mandatory, however, the method can in certain aspects include cure as well.

As used herein, the term “administering,” refers to the placement of stem cells as disclosed herein into a subject by a method or route which results in at least partial delivery of the cells to a desired site. Pharmaceutical compositions comprising the cells disclosed herein can be administered by any appropriate route which results in an effective treatment in the subject. Multiple compositions can be administered separately or simultaneously. Separate administration refers to the two compositions being administered at different times, e.g. at least 10, 20, 30, or 10-60 minutes apart, or 1, 2, 3, 4, 5, 6, 7, 8, 9, 10, 12 hours apart. One can also administer compositions at 24 hours apart, or even longer apart. Alternatively, two or more compositions can be administered simultaneously, e.g. less than 10 or less than 5 minutes apart. Compositions administered simultaneously can, in some aspects, be administered as a mixture, with or without similar or different time release mechanism for each of the components.

The terms “vascular-related degenerative neurological disease(s)” or “vascular-related degenerative neurological disorder(s)” refers to any neurological condition or disorder related to cranial vein dysfunction including but not limited to optic neuritis which is inflammation of the optic nerve in one or both eyes, neuromyelitis optica which is also known as Devic's disease, Lyme's Disease, migraine headache, cluster headache, vascular dementia, ischemic depression, ischemic stroke, motor neuron disease and multiple sclerosis.

As used herein, the terms “patient” or “patient population” refer to patients diagnosed as having a vascular-related degenerative disorder, in particular those having multiple sclerosis or at risk of developing multiple sclerosis. Diagnosing multiple sclerosis includes identification of symptoms of decreased neurological function. Symptoms include, but are not limited to, tremors, trembling in hands, arms, legs, jaw, and face; spasticity or rigidity of muscles, or stiffness of the limbs and trunk; bradykinesia, or slowness of movement; postural instability, or impaired balance and coordination; cognitive dysfunction leading to impairment of thinking, remembering, and reasoning skills including but not limited to amnesia, dysphasia, apraxia, agnosia; personality changes; depression; hallucinations; and delusions. Methods of diagnosing multiple sclerosis are known to those skilled in the art. The term “patient” includes any warm-blooded organism including, but not limited to, human beings, rats, mice, dogs, goats, sheep, horses, monkeys, apes, rabbits, cattle, etc.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal ora state government or listed in the U.S. Pharmacopeia or other generally recognized pharmacopeia for use in animals, and more particularly in humans. The term “carrier” refers to a diluent, adjuvant, excipient, or vehicle with which the stem cells are administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The terms “about” and “approximately” generally mean an acceptable degree of error for the amount measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “disease(s)” and “disorder(s)” have the same meaning.

### DETAILED DESCRIPTION

The present invention relates generally to the administration of stem cells to a patient having or at risk of developing or suspected to have a vascular-related degenerative neurological disorder. This administration can prevent or correct pathology associated with a vascular-related degenerative neurological disorder even to the point of stopping progression of the disease, improving neuronal activity to a sufficient level to reduce symptoms, or in some cases, return functions that have been lost to the disease.

Therefore, in one aspect of the present invention there is provided a method of treating a patient having or at risk of developing a vascular-related degenerative neurological disorder comprising administering a therapeutically effective amount of stem cells to the patient whereby the stem cells migrate to the cranial veins of the patient and reduce, or even in some cases surprisingly eliminate the symptoms associated with the disorder. Infused stem cells so delivered to dysfunctional venous vasculature, effectively stops or significantly reduces the leakage of neurotoxic blood proteins across the BBB. In many cases following this therapy, with the conditions that cause the disease mitigated, previously affected areas of the brain can recover function through the natural process of neuroplasticity. Research has confirmed that the central nervous system is plastic, malleable, and can recover after injury (Behrman A, 2006). This evidence has challenged older assumptions that brain damage due to disease or trauma was permanent. Evidence is compelling concerning locomotion especially with the group of patients whose data is contained in this document. The described method is useful in the treatment of vascular-related degenerative neurological diseases and/or disorders including but not limited to optic neuritis which is inflammation of the optic nerve in one or both eyes, neuromyelitis optica which is also known as Devic's disease, Lyme's Disease, migraine headache, cluster headache, vascular dementia, ischemic depression, ischemic stroke, motor neuron disease and multiple sclerosis.

Without being limited to the cause of the disease or disorder, a vascular-related degenerative disorder(s) or disease(s) is thought to be characterized by vascular dysfunction in the cranial veins of the patient leading to impaired venous drainage from the brain and degradation of the myelin sheath surrounding neurons in the brain of a subject. These diseases are treated by administration of certain sub-populations of stem cells which have the ability to differentiate into endothelium, myelin, neurons and other tissues. These cells will effectively migrate to sites of injury through chemotactic attraction.

Multiple sclerosis is a vascular-related degenerative neurological disorder of particular importance. Patients having MS may be identified by criteria establishing a diagnosis of clinically definite MS as defined by the workshop on the diagnosis of MS (Poser C M et al 1983) Revised criteria were developed in 2010. (Polman C et al 2011). Effective treatment of multiple sclerosis may be evaluated in several different ways. The following parameters can be used to gauge effectiveness of treatment.

Regardless of the treatment options and in an ideal evidence-based world, the most rigorous method of follow up, that of measuring comparative early histo-pathologic cortical and subcortical changes in neurogenesis is difficult. Recognizing that this approach to producing outcome data is challenging, researchers/scientists have had to establish outcome measures within a more appropriate domain to evaluate post-therapeutic functional outcomes.

Quantifiable clinician-observed data are now recognized as viable biomarkers for assessing changes in MS (Kaufman M et al 2000). The NIH Definitions Working Group defines a biomarker as a characteristic that is objectively measured and evaluated as an indicator of biologic processes, pathogenic processes or pharmacological response to a therapeutic intervention. It is also an exploratory tool that contributes to, and enables Go/No-go decisions in pre-clinical and early clinical phases of the development of prospective New Agents.

Walking speed (WS) is a valid, reliable, sensitive measure appropriate for assessing and monitoring functional status and overall health in a wide range of populations (McCuigan C et al 2004, Moti R W et al 2008). The Timed 25-Foot Walk (T25FW) is an important component of a standardized, quantitative, assessment instrument for use in clinical studies, particularly clinical trials of MS (Kaufman M et al, 2013, Schwid S R et al 2002). Change in T25FW is strongly correlated with disease impact (Keiseier B et al, 2012). It is an accepted, reproducible test for leg function adopted by the National Multiple Sclerosis Society's Advisory Committee on Clinical Trials of New Agents in Multiple Sclerosis. Widely available published instructions allow researchers from around the world the ability to reproduce the Timed 25-Foot Walk and follow the patients for the duration of any study.

This recognition of its utility has led to its designation as the 6th vital sign. (Middleton A., 2015) Walking speed tests can be performed in a variety of settings, making it a universal measure. The breadth of information provided by this assessment tool is not limited to inferences made based on a single time point. As a responsive measure (Barthuly A M et al 2012, Goldberg A et al 2011, Puthoff M L et al 2013), repeated WS tests can be used to monitor patients over time. For example, in a clinical setting a patient's WS at initial evaluation can be compared to their WS at reassessment post-therapy; or in a research setting WS may be used to determine changes over the course of a study and maintenance at follow up (Kaufman M et al 2000, Kaufman M et al 2013). In order to be confident that true change in WS has occurred, the difference between testing sessions needs to exceed the measurement error and natural variability that can occur with repeated measurements. A value that reflects this is the measure's minimal detectable change value (MDC is a statistical estimate of the smallest amount of change that can be detected by a measure that corresponds to a noticeable change in ability). If an individual's change in WS between testing sessions exceeds the MDC95, we can be 95% confident that a true change in WS has occurred. Our study utilizes the Timed 25-Foot Walk to record a specific functional outcome of MS patients treated with a novel therapy. The assessments are recorded immediately prior to treatment and again at 10 days post-therapeutic intervention. The walk tests reflected average improvements in 31 patients of values of >20%, a proven threshold for clinical significance (Hobart, J. 2013).

Kurtzke Expanded Disability Scale (EDSS) The Kurtzke Expanded Disability Scale was developed in the 1950s by Dr. John Kurtzke to measure the disability status of people with MS. The purpose was to create an objective approach to quantify the level of functioning that could be widely used by healthcare providers diagnosing MS. It is still the most popular scoring tool for MS. The disability rating scale is based upon neurological testing and examination, looking for abnormalities in functional systems. The EDSS is configured in relation to the functional systems it affects, including: pyramidal (motor functions like walking), cerebellar (coordination), brainstem (speech and swallowing), sensory (touch, vibration and pain), bowel and bladder functions, visual, mental, and any other (includes any other neurological findings due to MS). The EDSS provides a total score on a scale that ranges from 0 to 10. The first levels 1.0 to 4.5 refer to people with a high degree of ambulatory ability and the subsequent levels 5.0 to 9.5 refer to the loss of ambulatory ability. The range of main categories include (0)=normal neurologic exam; to (5)=ambulatory without aid or rest for 200 meters, disability severe enough to impair full daily activities; to (10)=death due to MS. Prospective surveys of patients at defined intervals allow for accurate self-evaluation because of the ease of making distinction between one level of function to the next with a much-reduced chance for bias impact (Kurtzke J F 1994).

The Multiple Sclerosis impact Scale (MSIS-29) may also be used to evaluate the effectiveness of the treatment. The MSS-29 Questionnaire is used for the assessment of health-related Quality of Life (QoL) in MS. The Multiple Sclerosis impact Scale (MSIS-29), a patient-based rating scale for MS developed in 2002, was predominantly developed from a community-based sample derived from the MS Society. The results demonstrate that the measurement properties of MSIS-29 in a range of hospital-based samples are very similar to those obtained in a community context and support the appropriateness of the instrument in a range of clinical settings. In contrast to the EDSS, MSIS-29 physical scores spanned nearly the full scale range and showed minimal floor and ceiling effects. This suggests that the floor and ceiling effects did not attenuate the correlations between scales. Furthermore, self-evaluation also provides little room to present bias into the outcome (McCuigan C et al 2004).

The Beck Depression inventory II (BDI-II) is another test. The Beck Depression inventory II is a 21-question multiple-choice self-report inventory that has been validated in screening for depression in MS. It assesses severity of depressed mood on a score from 0 to 63, reflecting less-to-more depressed mood, and is one of the most widely used instruments for measuring the severity of depression. Its development marked a shift among healthcare professionals, who had until then viewed depression from a psychodynamic perspective, instead of being rooted in the patient's own thoughts. In its current version the questionnaire is designed for patients aged 13 and over, and is composed of items relating to symptoms of depression such as hopelessness and irritability, cognitions such as guilt or feelings of being punished, as well as physical symptoms such as fatigue, weight loss, and lack of interest in sex. The study uses the BDI-II published in 1996. The BDI is actually universally used as an assessment tool by researchers in a variety of settings (Beck A T et al 1988).

Multidimensional Clinical Score Management is a multifactorial process and there are as many clinical scoring tools as there are clinical diseases. Weighting schemes are useful to quantify the relative importance of patient clinical items. For a medical analysis however, a Single global score is the most useful. This allows the establishment of a reliable method for relative quantification of items using an appropriate tridimensional clinical Scoring tool as a solution for weighted aggregation of three types of measurements including actual functionality, activities of daily living (ADL) and quality of life (Qol). With the objective of a global Score, the validation process for a combined clinical score necessitates the application of the correct scoring tools for any particular protocol. For MS, we chose an aggregate of the above three scales weighted equally for outcomes reflecting change as a result of treatment. Other diseases will use scales Commensurate to the symptoms and neurological deficits for that particular disease. Additionally, appearance of exacerbations on MRI (magnetic resonance imaging) is used as a parameter to gauge effectiveness of treatment for MS (Kurtzke J 1994).

In MS, exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are either mild, moderate, or severe according to changes in a Neurological Rating Scale (Sipe J et al 1984). An annual exacerbation rate and proportion of exacerbation-free patients are determined.

Therapy can be deemed to be effective if there is a statistically significant difference in the rate or proportion of exacerbation-free or relapse-free patients between the treated group and the placebo group for any of these measurements. In addition, time to first exacerbation and exacerbation duration and severity may also be measured. A measure of effectiveness as therapy in this regard is a statistically significant difference in the time to first exacerbation or duration and severity in the treated group compared to control group. An exacerbation-free or relapse-free period of greater than one year, 18 months, or 24 months is particularly noteworthy.

Clinical measurements include the relapse rate in one and two-year intervals, and a change in EDSS, including time to progression from baseline of 1.0 unit on the EDSS that persists for six months. On a Kaplan-Meier curve, a delay in sustained progression of disability shows efficacy. Other criteria include a change in area and volume of T2 images on MRI, and the number and volume of lesions determined by gadolinium enhanced images. MRI can be used to measure active lesions using gadolinium-DTPA-enhanced imaging as outlined in the 2010 revised McDonald criteria (Polman C et al 2011), or the location and extent of lesions using T2-weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of MS lesions can be determined by radiologists. Areas of lesions can be outlined and summed slice by slice for total lesion area. Three analyses may be done: evidence of new lesions, rate of appearance of active lesions, percentage change in lesion area (Paty D W et al 1993).

Improvement due to therapy can be established by a statistically significant improvement in a patient compared to baseline or in a treated group versus a placebo group. Exemplary symptoms associated with multiple sclerosis, which can be treated with the methods described herein, include: optic neuritis, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadriplegia, spasticity, dysarthria, muscle atrophy, spasms, cramps, hypotonia, clonus, myoclonus, myokymia, restless leg syndrome, footdrop, dysfunctional reflexes, paraesthesia, anesthesia, neuralgia, neuropathic and neurogenic pain, L'hermitte's sign, proprioceptive dysfunction, trigeminal neuralgia, ataxia, intention tremor, dysmetria, vestibular ataxia, vertigo, speech ataxia, dystonia, dysdiadochokinesia, frequent micturition, bladder spasticity, flaccid bladder, detrusor-sphincter dyssynergia, erectile dysfunction, anorgasmia, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoff symptom, gastroesophageal reflux, and sleeping disorders.

In addition to or prior to human studies, an animal model can be used to evaluate the efficacy of treatment. An exemplary animal model for multiple sclerosis is the experimental autoimmune encephalitis (EAE) mouse model, e.g., as described in (Tuohy V K 1988, Sobel R A et al 1984, Traugott U 1989) Mice can be administered cells as described herein. Then the mice are evaluated for characteristic criteria to determine the efficacy of using the agent in the model.

Accordingly, in a preferred embodiment of the present invention there is provided a method of treating a patient having multiple sclerosis comprising intravenously administering a therapeutically effective amount of stem cells to the patient such that the cells migrate to the cranial veins of the patient.

Stem Cells and Stem Cell Populations

Many chronic and currently untreatable diseases in humans, mostly age-related, arise from the loss or malfunction of specific cell types in the body. In the same way that donated organs such as hearts, lungs or kidneys are often used to replace damaged or dysfunctional ones, stem cells can be used to replace certain tissues that have become diseased or worn out over time. Sometimes, under specific circumstances, the patient's own stem cells can even be used, transplanted from one place to another in the body, or even grown outside in the body in a lab to multiply them before transplanting even a greater number (Kirouac D C 2008).

There are only 2 diseases that are approved by the FDA for stem cell treatment in the US. These are lymphoma and leukemia and their sub-types, all blood-borne malignancies. Clinicians in the US do not have the legal ability to practice or promote regenerative medicine (therapies using stem cells) in any other area of medicine no matter how promising results are, and funding for stem cell trials is meager compared to the amount spent by drug companies (the major sponsors for all medical research world-wide) on research into new proprietary drugs. There are many reasons for this, including historical, political and financial (Lodi D et al 2011).

The Nature of Stem Cells

Stem cells have two important and unique characteristics: Firstly, they are biological cells capable of renewing themselves through cell division. They can replicate themselves to produce more stem cells, and differentiate or transform into specialized cells required for growth and maintenance of living tissue. When a stem cell divides, each new cell has the potential either to remain a stem cell or to differentiate into other kinds of cells that form the body's tissues and organs. Stem cells can theoretically divide without limit to replenish other cells that have been damaged. Secondly, under certain controlled conditions, stem cells can be induced to become tissue- or organ-specific cells with special functions. They can then be used to treat diseases affecting those specific organs and tissues. (National Institutes of Health. Stem cell basics. April 2009. Available at: http://stemcells.nih.gov/info/basics/Pages/Default.aspx. Accessed Aug. 8, 2012).

In mammals and humans there are two broad types of stem cells: embryonic stem cells, which are formed during the blastocyst (early formation) phase of embryological development, or adult stem cells. The advantage of embryonic stem cells is that they are pluripotent, meaning they can develop into any of the more than 200 cell types found in the body, providing the potential for a broad range of therapeutic applications (Yu J, et al 2008). The second type is adult stem cells which are found in various tissues of the body. In a preferred embodiment, the process described herein concerns itself only with adult stem cells. In the human body, adult stem cells have been identified in many organs and tissues including the brain, bone marrow, peripheral blood, blood vessels, skeletal muscle, skin, dental pulp, gut, liver, ovarian epithelium and testis. They are thought to reside in a specific area of each tissue called a stem cell niche (Shi S et al 2003).

Stem cells are further categorized for other properties and their potency or potential to become cells of various tissues. There are many places in the body where adult cells can be found. The bone marrow contains at least two main kinds of stem cells. One population, called hematopoietic stem cells, forms all the types of blood cells in the body. A second population, called bone marrow mesenchymal stem cells (also called stromal cells or just MSC's, make up a smaller proportion of the stromal cell (connective tissue cell) population in the bone marrow and can generate bone, cartilage, and fat cells that support the formation of blood and fibrous connective tissue (Kresbach P H et al 1999). Although MSCs are mostly found in bone marrow, similar cells have been isolated from tissues such as peripheral blood, cord blood, umbilical cord derived Wharton's jelly, adipose tissue, amniotic fluid, placenta, fetal tissues, dental pulp, periosteum, synovial fluid and membrane, articular cartilage, skeletal muscle and dermis, and lungs. However, it is not yet clear how similar or dissimilar MSCs derived from bone marrow are from those derived from other human tissues. MSCs are localized in the vascular niche in bone marrow and are also found as MSC-like cells around adult vessels called pericytes.

Pericytes are another type of stem cell found in the bone marrow and other body tissues. Pericytes are contractile cells that wrap around the endothelial cells of capillaries and venules throughout the body. These cells form layers of tissue in blood vessels. Pericytes play a crucial role in the formation and functionality of the selectively permeable space between the circulatory system (of blood vessels) and the central nervous system. This space is known as the blood-brain barrier or BBB and the focus on its treatment is critical to the process described herein (Bergers G et al 2005, Oswald et al 2004, Wagey R et al 2014, Watt et al 2013).

The use of the patient's own stem cells as well as the technological development of stem cell lines that can replicate many tissues of the human body is an important advancement for medical science. A long-standing and common use of adult stem cells is for the treatment of lymphomas and leukemias. These stem cells come from matching donor stem cells and are known as allogeneic (from individuals of the same species) stem cells. For many other diseases, stem cell research has the potential to revolutionize the practice of medicine and improve the quality of life for many people with chronic diseases. Given the enormous promise of stem cell therapies for so many devastating diseases, most scientists believe that it is important to simultaneously pursue all avenues of research in order to investigate the best sources and the best uses of many stem cell types (Brazzini A. 2010).

One promising source of stem cells is from umbilical cord tissue derived Wharton's jelly, taken immediately after birth. In a developing embryo, stem cells can differentiate into all of the necessary specialized cells; the ectoderm, the mesoderm and the endoderm. These stem cells maintain the normal turnover of the cells of regenerative organs such as blood, skin or intestinal tissues. In adult organisms, stem cells and progenitor cells act as a repair system for the body replenishing adult tissues. Stem cells and progenitor cells are similar but progenitor cells come from a further stage of cell differentiation that is more specific than stem cells and they are already being encouraged by environmental factors to differentiate into their target cells. (Lodi D et al 2011).

True stem cells have an unlimited capacity for self-renewal whereas the progenitor cells are limited in their capacity for self-renewal (potentially up to 3-4 times only). Most progenitor cells are described as oligopotent, meaning they have the abilityto differentiate into several different cell types, and may be compared to adult stem cells. Both progenitor and adult stem cells share many properties. Progenitor cells act as a repair system for the body and may remain quiescent (non-dividing) for long periods of time until they are activated by a normal need for more cells to maintain tissues, or by disease or tissue injury. The majority of the progenitor cells lie dormant or are minimally active in the tissue in which they reside. They exhibit slow growth and their main role is to replace cells lost by normal attrition. In case of tissue injury, damaged or dead cells, progenitor cells are activated. Growth factors and cytokines are two substances that trigger the progenitors to mobilize toward the damaged tissue. At the same time, they start to differentiate into the target cells. Not all progenitors are mobile and are naturally situated near the tissue of their target differentiation. When the cytokines, growth factors, and other cell division enhancing stimulators take on the progenitors, a higher rate of cell division is induced, leading to the repair and recovery of the tissue. The characterization or the defining character of progenitor cells, in order to separate them from others, is based on the different cell markers inherent within, and on the cell surface, rather than their morphological appearance. (Wagey R et al 2014).

There are many different cell types and many different sources of stem cells in the body. Some of these cells are known to migrate to the olfactory bulb and differentiate further into specific types of neural cells (astrocytes); bone marrow stromal cells are often classified as stem cells due to their high plasticity and potentiality, and unlimited capacity for self-renewal; periosteum (vascular connective tissue surrounding bones) contains progenitor cells that develop into osteoblasts (which become bone) and chondrocytes (which become cartilage); pancreatic progenitor cells are among the most studied progenitors.

There are three known accessible sources of autologous adult stem cells in humans: bone marrow which requires extraction by harvesting, that is by aspirating from a flat bone, typically the iliac crest, sternum or the femur; adipose tissue (lipid or fat cells), which requires extraction by liposuction; and blood, which requires extraction through apheresis wherein blood is drawn from the donor (similar to a blood donation), and passed through a machine that extracts the stem cells and returns other portions of the blood to the donor.

Of all stem cell types, autologous, meaning from the patient, harvesting involves the least risk. By definition, autologous cells are obtained from the patient's body, and may be used for an immediate procedure just as they may be banked for future elective surgical procedures. Stem cells can now be artificially grown and transformed (differentiated) into specialized cell types with characteristics consistent with cells of various tissues such as muscles or nerves. Embryonic cell lines and autologous embryonic stem cells have also been proposed as promising candidates for future therapies (Lodi D et al 2011).

The classical definition of a stem cell requires that it possess two properties: Self-renewal: the ability to go through numerous cycles of cell division while maintaining the undifferentiated state and potency: the capacity to differentiate into specialized cell types. In the strictest sense, this requires stem cells to be either totipotent or pluripotent—to be able to give rise to any mature cell type, although multipotent or unipotent progenitor cells are sometimes referred to as stem cells. Apart from that stem cell function is regulated in a feedback mechanism. Potency specifies the differentiation potential (the potential to differentiate into different cell types) of the stem cell.

Stem Cell Potentiality or Potency

Cell potency is a cell's ability to differentiate into other cell types. The more cell types a cell can differentiate into, the greater its potency. There are several general categories of potency: totipotent (a.k.a. omnipotent) stem cells can differentiate into embryonic and extra-embryonic cell types. Such cells can construct a complete, viable organism. These cells are produced from the fusion of an egg and sperm cell. Cells produced by the first few divisions of the fertilized egg are also totipotent. Pluripotent stem cells are the descendants of totipotent cells and can differentiate into nearly all cells i.e. cells derived from any of the three germ layers (ectoderm, endoderm ad mesoderm). Multipotent stem cells can differentiate into a number of cell types, but only those of a closely related family of cells. Oligopotent stem cells can differentiate into only a few cell types, such as lymphoid or myeloid stem cells. Lastly, unipotent stem cells can produce only one cell type, their own but have the property of self-renewal, which distinguishes them from non-stem cells (e.g. progenitor cells) which cannot self-renew.

Properties of Allogeneic Stem Cells and induced Pluripotent Stem Cells

Allogeneic cells are stem cells collected from a matching donor and transplanted into the patient to suppress the disease and restore the patient's immune system. Allogeneic transplant is called an allograft. This procedure is most commonly used to treat blood-related diseases such as non-Hodgkin's Lymphoma, Hodgkin's Lymphoma, leukemia and multiple myeloma. An allogeneic stem cell transplant is different from an autologous stem cell transplant which uses the cells from one's own body (Passweg J R et al 2012).

Perhaps more than any other regenerative medicine methodology, the use of induced pluripotent stem cells or iPSCs derived from somatic cells may represent the most powerful tool as a therapy in chronic, degenerative diseases. Induced pluripotent stem cells (iPSCs) are a type of pluripotent stem cell that are generated from an adult cell by reactivating a small number of genes and turning the mature specialized cells back into pluripotent stem cells (a process called reprogramming). Although the mechanism by which these genes cause adult cells to become pluripotent is not yet fully understood, (a process known as epigenetics), the technique holds great promise for stem cell research and regenerative medicine. Due to their great similarity to human Embryonic Stem Cells (hESCs), iPSCs are of great interest to the medical and research community. It presents a new opportunity to generate disease specific stem cell lines in order to study certain disease conditions and to screen promising new drugs. The iPSCs have similar therapeutic implications and applications as hESCs but without the controversial use of embryos in the process, a topic of significant ethical debate. While the discovery of iPSCs is a significant breakthrough, as was recently acknowledged when the discoverer, Shinya Yamanaka (Yamanaka S 2012), was awarded the 2012 Nobel Prize for Medicine or Physiology, they are only just entering clinical trials. In fact, at present, they have not yet been approved for clinical stage research in the United States. (Riesman M, et al. 2014).

In certain embodiments of this aspect of the invention, the stem cells used in the methods and compositions provided herein have the capacity, and are selected for their capacity, and in the therapeutic numbers required to differentiate to become endothelial tissue, the tissue that has become dysfunctional within the venous vascular system in MS. Many of the stem cell types present in bone marrow are potentially suitable for this aspect of the invention because many can differentiate to become the tissues required for repair and maintenance of blood vessels under the stressful conditions that exist in the disease state. There are bone-marrow derived adult stem cells of many types that can renew themselves and/or can differentiate to yield specialized cell types of tissues or organs required to return the disease state to homeostatic balance, or the ability for the body to restore a functional, stable internal environment (Kresbach PH et al 1999). Examples include hematopoietic stem cells and mesenchymal stem cells, and subcategories of these cells such as adipocyte cells, fibrocyte cells, endothelial cells, pericyte cells, and monocyte cells listed herein.

With respect to a preferred embodiment of the methods and compositions of the present invention described herein, the source of the stem cells used for this invention are harvested from the patient's own bone marrow (BM); these are otherwise known as autologous stem cells. Bone marrow is the flexible tissue in the interior of bones. There are two types of bone marrow; red marrow (Latin: medulla ossium rubra), which consists mainly of hematopoietic tissue, and yellow marrow (Latin: medulla ossium flava), which is mainly made up of fat cells (Bain B 1996).

Recently, bone marrow-derived cells that developed into pericytes enveloping the growing vasculature in injured tissues were identified. One of the first studies, which proposed the existence of bone marrow-derived pericyte progenitors, showed that CD11b and CD45 hematopoietic cells, expressing the pericyte marker NG2, were detected in close proximity to blood vessels. The existence of bone marrow-derived pericyte progenitor cells (PPCs) was further substantiated by the identification of PDGFR-β pericyte progenitors (PPPs) in an endogenous mouse model. Given that only subsets of PPPs are recruited from the bone marrow (Song, S, 2005, Ozerdem, U, 2005), it is conceivable that PPPs can also be attracted to angiogenic vessels from the local environment or become activated within the injured tissue.

There is emerging evidence that bone marrow-derived cells play a crucial role in the formation of blood vessels in the adult by incorporating into the growing vasculature, localizing adjacent to it, or enveloping the vasculature to serve as perivascular support cells. In MS the venous smooth muscle cells break down at the tight junctions and cannot recover as the vein becomes dysfunctional. Without being limited to any theory of causation, aging may play a role as the body loses red marrow and not enough cells can be recruited. Another cause may be the result of anomalous persistent hemodynamic stressors. Another cause may involve defects in PDGF-B/PDGFR-β signaling affecting pericytes and endothelial cells, leading to an abnormal vasculature. It is therefore logical that the local introduction of a therapeutic population of bone marrow cells containing PPCs and endothelial cells support reactivation of existing endothelial cells to interact with vessel basement membrane surfaces within the injured tissue through paracrine signaling as well as the direct repair mechanisms of the infused cells (Lamagna C, 2006).

Cluster of Differentiation

Cells can be further characterized by their cluster of differentiation or CD attributes. Briefly, this is a number indicating the identification of cell surface molecules providing targets for the immunophenotyping of cells (a technique used to study the proteins expressed by a cell). Many functionally different populations of cells are microscopically indistinguishable from one another. CD markers allow detection of the surface proteins that designate different functions for visibly similar cells. The numbers, of which there are more than 370, with more being found every year, have no specific meaning with regard to their function. The cluster of differentiation number (CD) is simply a naming convention that immunologists use, because over time, a seemingly endless number of cell surface proteins were being discovered on the cells within our bodies. Essentially CD proteins are named in the order that they were discovered (meaning CD1 was discovered before CD10) (IUIS-WHO Nomenclature Subcommittee 1984).

In terms of physiology, CD molecules can behave in numerous ways, often acting as receptors or ligands (the molecule that activates a receptor) important to the function of the cell. A signal cascade is usually initiated altering the behavior of the cell, known as, cell signaling. Cell signaling can be classified as mechanical and/or biochemical based on the type of signal. These energy stimuli can both be assimilated and responded to by cells (Miller C J et al 2013). Cell signaling is part of a complex system of communication that controls basic activities of cells and regulates their actions. The ability of cells to discern and correctly respond to their microenvironment from both within and without is crucial to their maturation, tissue repair and regeneration, and immunity. The normal activity of cells, as complex as it appears to be, creates homeostasis, which is the regulation of systems to maintain a constant and healthful condition in the maintenance of life. Errors in cellular information processing are responsible for diseases such as cancer, or the autoimmune responses we see in Multiple Sclerosis. (Miller C J et al 2013).

Biochemical signals are molecules in the form of proteins, lipids, ions and gases. These signals can also be categorized based on the distance between signaling and responder cells. Signaling within, between, and among cells is subdivided into various classifications. In the case of our process, it is paracrine signaling. Paracrine signaling is a form of cell-to-cell communication in which a cell produces a signal to induce changes in nearby cells, altering the behavior and/or differentiation of those cells. Signaling molecules known as paracrine factors diffuse over a relatively short distance (local action), as opposed to endocrine (hormones which travel considerably longer distances via the circulatory system). Neurotransmitters in the brain represent an example. Cells that produce paracrine factors secrete them into the immediate extracellular environment. Factors then travel to nearby cells in which the gradient of factor received determines the outcome. The exact distance that paracrine factors can travel is not certain, but in any therapy it is better to concentrate treatment on the local area of interest (Lodish H et al 2000).

A common and well-known example is a CD34+ cluster of differentiation first described independently by Civin C I et al, 1984, in a cell surface glycoprotein, which functions as a cell-to-cell adhesion factor. It may also mediate the attachment of stem cells to bone marrow extracellular matrix or directly to stromal cells. In adult humans, cells expressing CD34+ are normally found in bone marrow as hematopoietic cells, a subset of mesenchymal stem cells, endothelial progenitor cells, and endothelial cells in blood vessels. CD34+ cells may be isolated from bone marrow using established methods such as immunofluorescence in the laboratory. Infusion of CD34+ hematopoietic stem cells has been clinically applied to treat various diseases including both nerve injury (Srivastava A, 2010), and vascular diseases (Vidyasagar D D, 2012).

In another embodiment of this aspect of the invention, the stem cells used in the methods and compositions provided herein have the capacity, and are selected for their capacity, to differentiate to become neural cells or neurons, which are required to recover function that has been lost due to the MS disease process. Stem cells suitable for this aspect of the invention include cord tissue, preferably allogeneic cord tissue, which include pluripotent stem cells that are able to specialize into neurons, as well as have the ability to self-renew, embryonic stem cells, particularly embryonic Stem cells derived from blastocyst stage, and reprogrammed cells or induced pluripotent stem cells (iPSC), so called because they are manipulated by specific gene encoding transcription factors that convert any adult cells into pluripotent stem cells. This characteristic allows these cells to propagate indefinitely, as well as give rise to every other cell type in the body including neurons.

Preferably, the stem cells are bone marrow stem cells, that is, mesenchymal stem cells or hematopoietic stem cells, otherwise known as bone marrow-derived mononuclear cells (BMMNCs). The stem cells can be derived from an allogeneic, autologous or even an ex vivo source. Most preferably the stem cells are removed from the patient to be treated such that the stem cells are autologous stem cells. In the most preferred embodiment the stem cells are autologous bone marrow-derived stem cells (BMMNCs).

Removal and Isolation of Stem Cells

In one embodiment a source of cells derived from body fat tissue or adipose tissue may be used, although populations of the cells required to succeed in the performance of the protocol herein described are not as numerous from that source. The isolation of adipose-derived stem cells (ASCs) by way of liposuction wastes typically 8-10 hours of continuous intense effort, making it a labor-intensive endeavor and increasing the risk of culture contaminations due to excessive handling (Francis M, 2010).

Stem cells may be removed from the body of a patient in ways known in the art. In a preferred embodiment of this aspect of the invention the stem cells useful in the methods and compositions described herein can be obtained, e.g., by removing them from particular bones in a patient's body, among them the femur and the sternum but preferably from the iliac crest because it is the largest reservoir of bone marrow in the human body. Ina preferred embodiment, a bore aspiration needle (Becton Dickinson) is used under aseptic technique to bore into the bone marrow within a bone and remove a bone marrow aspirate. In a preferred embodiment of this aspect of the invention autologous bone marrow mononuclear cells (BMMNCs) are isolated from the entire sample of bone marrow via centrifuge. A reduced sample of bone marrow contains a high population of Cluster of Differentiation 34 also known as CD34+ progenitor cells. CD34+ progenitors are frequently isolated from bone marrow-derived mesenchymal stem cells (BMMSCs) and used for a range of research experimentations and in clinical settings for treatments for many diseases, including liver cirrhosis, peripheral vascular disease, and other degenerative diseases. The stem cells are isolated from a population of bone marrow-derived adult stromal cells.

In various embodiments, the removed stem cells, contained within the bone marrow aspirate obtained from a patient's body are at least 50%, 60%, 70%, 80%, 90%, 95%, 99% or at least 99.5% of said population of cells.

Processing of Bone Marrow Aspirate

Aspirate collected from the patient is added to EDTA, anticoagulant and is processed in the appropriately equipped laboratory. After proper mixing (inverting thetube gently several times), the aspirate sample should be pipetted into a 1 mL Wintrobe tube, assuring that there are no air bubbles. Wintrobe tubes are placed in a centrifuge and spin at 2800-3000 rpm for approximately 8 minutes. After centrifugation, the sample is separated into four distinct layers. From the bottom to the top of the aspirate in the Wintrobe tube are erythrocytes (E)/red blood cells (RBCs), myeloid-erythroid (M-E) cells (buffy coat), plasma (P), and the fat and perivascular (F-PV) cells.

The bone marrow stem cells for use in the present invention may be isolated from the bone marrow aspirate by any separation technique known to those of skill in the art. In a preferred embodiment the stem cells are isolated through centrifugation. The aspirate is centrifuged (Benchtop ST 16) at 2800 RPM (@850 g) for 8 minutes to obtain a concentrated phase containing mononuclear cells (MNC). The plasma is separated and discarded from the mononuclear fraction in both volumes. Aliquots are taken from the above sample to measure total nucleated cell count, MNC count, and viability testing is done using a (Countess II Automated) cell-counter to assess viability of cell tissue. Final yield of all cell types. The cell-counter assesses the viability (whether cells are living or dead), it doesn't have anything to do with isolating them. Normally cell populations are found to be 80%-95% viable (living) but reinfusion at levels above 45% are routinely performed in clinical studies. Cell types are indeed derived from BMA and there are many types of cells, particularly pericytes found in abundance in bone marrow, but many are useful for the purpose of repairing the endothelial lining of the leaking veins for reinfusion is between 300 million-1 billion cells of all cell types present in bone marrow, and depending on the individual patient.

The populations of isolated stem cells described above, and populations of stem cells generally, can comprise about, at least, or no more than, 300 million to 1 billion, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more of the isolated stem cells. In a preferred embodiment the isolated stem cells are autologous bone marrow stem cells.

Growth in Culture

Alternatively, the stem cells used in the method of the present invention may be grown ex vivo in cell culture. The growth of the cells, e.g., the autologous bone marrow stem cells described herein, as for any mammalian cell, depends in part upon the particular medium selected for growth. Under optimum conditions, cells typically double in number in 3-5 days. During culture, the placental cells provided herein adhere to a substrate in culture, e.g. the surface of a tissue culture container (e.g., tissue culture dish plastic, fibronectin-coated plastic, and the like) and form a monolayer. The process must be undertaken in a clean environment not less than ‘class 1000’ for effective culture outcomes.

A nutrient medium or culture medium is a liquid or gel designed to support the growth of microorganisms, cells, or small plants. Cell culture media generally comprise an appropriate source of energy and compounds which regulate the cell cycle. A typical culture medium is composed of a complement of amino acids, vitamins, inorganic salts, glucose, and serum as a source of growth factors, hormones, and attachment factors. In addition to nutrients, the medium also helps maintain pH and osmolality.

In vitro cultured cells are subjected to an environment whose main components are the medium, the atmosphere, the substrate and cell-to-cell interactions. Each of these components participates in a highly complex network of signaling pathways culminating in the determination of the fate of the stem cells (Van Der Sanden B, 2010).

The fact that cell culture medium is also named cell growth medium illustrates a point. It rapidly becomes clear that culture medium influences cell fate and acts not onlyas a feeder but also as an instructor. This point is particularly relevant for stem cells in culture which always balance between self-renewal or cell differentiation. Therefore, devising fully defined media able to maintain sternness which is the ability of the stem cell to have the capacity to self-renew and generate differentiated cells, or alternatively to drive differentiation towards well-defined phenotypes, is a point of major concern for stem cell science. Because stem cells are diverse, a universal optimal stem cell culture medium does not exist, and distinct stem cell types may require different culture conditions. This is true for all stem cell types and the culture that suits one may not suit another. Research into the effects of these four essential components of in vitro culture is an actively growing field of investigation with remarkable applications both for our understanding of stem cell biology and regenerative medicine. (Peerani Ret al, 2010; Voog J et a12010).

Stem Cell Collection Composition

Stem cells can be collected and isolated according to the methods provided herein. Generally, stem cells are obtained from the patient using a physiologically-acceptable solution, e.g., a stem cell collection composition. Examples of stem cell collection compositions are described in detail in related U.S. Provisional Application No. 60/754,969, filed Dec. 29, 2005.

The stem cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, HDMEM, etc.), and the like (Sotiropoulos P A et al 2006).

The stem cell collection composition can comprise one or more components that tend to preserve stem cells, that is, prevent them from dying, or delay the death of the stem cells, reduce the number of stem cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The stem cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

Culture Media

The stem cells of the present invention can be cultured in any medium, and under any conditions, recognized in the art as acceptable for the culture of stem cells. Preferably, the culture medium comprises serum. Stem cells can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 10% FBS, EGF, and heparin; α-MEM (minimal essential medium) comprising 10% FBS, GlutaMAX™ and gentamicin; DMEM comprising 10% FBS, GlutaMAX™ and gentamicin, etc. A preferred medium is DMEM-LG/MCDB-201 comprising 2% FBS, ITS, LA+BSA, dextrose, L-ascorbic acid, PDGF, EGF, and penicillin/streptomycin.

The media used herein contains Dulbecco's-modified Eagle's Medium-Low Glucose (DMEM-LG, GIBCO Invitrogen; Carlsbad, Calif.; http://www.invitrogen.com) supplemented with GlutaMAX™ 2 mM (L-alanyl-L-glutamin; GIBCO Invitrogen), penicillin 10 U/ml, streptomycin 100 μg/ml (both from Biochrom), and 10% fetal calf serum (FCS). In order to identify factors able to lead differentiation of stem cells towards cells of neural lineage, we isolated cells from human adult bone marrow (BMSC). Cells were treated with: TPA, forskolin, IBMX, FGF-1. MSC cultures grow in an incubator (T/Scientific) at 37° C. in 5% CO2.

Preservation of Stem Cells

Stem cells of the present invention can be preserved, that is, placed under conditions that allow for long-term storage, or conditions that inhibit cell death by, e.g., apoptosis or necrosis. The stem cells can be preserved using, e.g., a composition comprising an apoptosis inhibitor, necrosis inhibitor and/or an oxygen-carrying perfluorocarbon, as described in related U.S. Provisional Application No. 60/754,969, entitled “Improved Composition for Collecting and Preserving Placental cells and Methods of Using the Composition” filed on Dec. 25, 2005. DSMO can be added as a cryoprotectant added to cell media to reduce ice formation and thereby prevent cell death during the freezing process. In one embodiment, provided herein is a method of preserving a population of stem cells comprising contacting said population of stem cells with a stem cell collection composition comprising an inhibitor of apoptosis and an oxygen-carrying perfluorocarbon, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase inhibitor. In another specific embodiment, said inhibitor of apoptosis is an INK inhibitor. In a more specific embodiment, said JNK inhibitor does not modulate differentiation or proliferation of said stem cells. In another embodiment, said stem cell collection composition comprises said inhibitor of apoptosis and said oxygen-carrying perfluorocarbon in separate phases. In another embodiment, said stem cell collection composition comprises said inhibitor of apoptosis and said oxygen-carrying perfluorocarbon in an emulsion. In another embodiment, the stem cell collection composition additionally comprises an emulsifier, e.g., lecithin. In another embodiment, said apoptosis inhibitor and said perfluorocarbon are between about 0° C. and about 25° C. at the time of contacting the stem cells. In another more specific embodiment, said apoptosis inhibitor and said perfluorocarbon are between about 2° C. and 10° C., or between about 2° C. and about 5° C., at the time of contacting the stem cells. In another more specific embodiment, said contacting is performed during transport of said population of stem cells. In another more specific embodiment, said contacting is performed during freezing and thawing of said population of stem cells.

After collection of the cells, in a preferred embodiment the stem cells of the present invention are administered to a vein in the body of the patient. In a preferred embodiment of this aspect of the invention, the stem cells described above may be administered to an internal jugular vein, azygous vein or vertebrate vein of the patient. In an even more preferred embodiment the stem cells are administered to the internal jugular veins.

The cells are administered intravenously by any appropriate technique. Preferably a catheter is used to administer the cell. In an even more preferred embodiment of the present invention the catheter is a balloon tipped catheter. Balloon tipped catheters are capable of expanding and thereby occluding the vein in which the cells are administered to such that the cells are maintained distally with respect to the point of occlusion. In this context distal or distally means further away from the center of the body such or upstream from the internal jugular veins at the point of the cranial sigmoid sinus. The cranial sigmoid sinus is a venous structure at the base of the brain connecting the jugular veins to the cranial veins. In the process described herein, catheter-delivered stem cells deposited into the sigmoid sinus via retrograde flow are delivered at a relatively low pressure to the blood brain barrier (BBB). Once infused, stem cells adhere tightly to the endoluminal walls of the sigmoid sinus area from where they access the BBB in the cranial veins through paracrine signaling, translocation, and other chemotactic factors.

Balloon tipped catheters suitable for use in the present invention include a 5-F Check-Flo catheter. The catheter is inserted into a vein including the right or left femoral vein, preferably the right femoral vein. In a more preferred embodiment a guide wire is used to advance through a needle punctured into the femoral vein. A balloon catheter is then introduced into the vein over the wire. The catheter is moved to ultimately insert into the right atrium and into the superior vena cava. This is known as fluoroscopically-guided internal jugular catheterization. From the superior vena cava, the physician operator can access the veins that drain the central nervous system, including the internal jugular veins, the azygous vein, vertebral veins, and others. Preferably, the stem cells are administered into the internal jugular veins, the azygous vein or the vertebral vein. Even more preferably, the stem cells of the present invention are administered to the internal jugular vein of the patient. Fluoroscopically-guided internal jugular catheterization has the advantage of providing real time visualization in order to achieve precision in catheter and catheter-tip placement within the vein. In a preferred embodiment of this aspect of the invention lines and introducer sheaths are placed in the blood vessels with fluoroscopic guidance so that the Interventional Specialist may ensure the expected venous trajectory is followed during insertion.

In a more preferred embodiment of this aspect of the invention, the operator injects a radio-opaque contrast agent into the blood vessel through the catheter to verify venous obstructions and/or to observe blood flow characteristics in real time fluoroscopy. Cervical and intracranial veins are studied to map out the anatomical sinuosity and to record anatomical variants. The anatomy of cervical veins across individuals with MS varies greatly. The clinical and histologic features of tortuous cervical veins in MS are the result of disruption of the normal structure of the venous wall as a result of remodification of the extracellular matrix (ECM) in response to increased venous distention and altered hemodynamic stressors. Images acquired through catheter venography can be archived for later analysis and become part of the patient record. If an obstruction, occlusion or tortuosity is found in the right or left internal jugular vein, or azygous vein as it is in most cases in MS, the catheter is moved over the lesion area and inflated (Venoplasty). By coupling diagnostic confirmation and treatment, catheter movement and relocation is minimized, thus reducing risk. The cells are selectively transfused to the jugular vein of the patient by a balloon tipped catheter.

In balloon angioplasty, often called venoplasty in veins, vein obstruction is synthesized (artificially induced) by inflating a cylindrically-shaped balloon inside the vein for a short period of time. The balloon, tightly wrapped around a catheter shaft to minimize its profile, is inserted through the skin at the femoral vein puncture site (or working end) and into the narrowed section of the vessel. There are a range of catheters with various dimensions that are suitable for use in the present invention which can be used depending upon the pathophysiology of the patient being treated. Preferably, diagnostic pancerebral angiography may be performed with any suitable balloon catheter, preferably a Cobra Soft Radiopaque Tip Balloon Dilatation Catheter 5F 038 inch 120 cm vascular catheter. Compliant-type balloon catheters are employed because greater flexibility in these catheters allows for insertion in the narrow arch of the azygous vein, if required. Balloon dimensions range, depending on individual pathology, preferably from 6-10 mm in width and preferably 2-6-cm in length inflated preferably to a maximal pressure of about 6 atm.

The balloon is inflated by any appropriate means, preferably with saline forced through a syringe. The saline exerts pressure on a tortuous vein and/or causes blood to temporarily flow in an opposite direction, concomitant to the stem cell infusion. For retraction, a vacuum is pulled through the balloon to collapse it and the blood again flows normally. Inflations are maintained for as long as necessary, preferably between about10 seconds and 2 minutes, more preferably for about 20 seconds and 1 minute and most preferably for about 30 to about 60 seconds at a time and repeated as necessary, for example 1 to 20 times, preferably 5 to 10 times, most preferably to infuse the entire volume of stem cell fluid mixture. If a lesion is present, the balloon maintains a fixed position over the lesion as the process is repeated.

Thus, another aspect of the present invention is directed to a balloon tipped catheter having stem cells releasable associated therewith wherein after release of the stem cells the lumen of the catheter occludes the vein proximally to the release point of the stem cells such the stem cells migrate to the cranial veins of the patient and are maintained in a local retrograde flow. In this context retrograde flow means flow against the direction of the flow of the majority of the components in that area, e.g. the vascular fluid. Releasably associated therewith means that the stem cells of the present invention are able to be carried by the catheter and easily released therefrom, for example, by dispelling them under pressure.

Another aspect of the present invention relates to pharmaceutical compositions comprising the stem cells of the present invention. In a preferred embodiment of this aspect of the invention populations of stem cells, or populations of cells comprising stem cells, can be formulated into pharmaceutical compositions for use in vivo. Such pharmaceutical compositions comprise a population of stem cells, or a population of cells comprising stem cells, in a pharmaceutically-acceptable carrier, e.g., a saline solution or other accepted physiologically-acceptable solution or preferably a pharmaceutical medium for in vivo administration. Pharmaceutical compositions provided herein can comprise any of the stem cell populations, or stem cell types, described elsewhere herein. The pharmaceutical compositions comprise, for example, autologous bone marrow stem cells.

The pharmaceutical compositions provided herein comprise any therapeutically effective amount of stem cells. In a specific embodiment, a single unit dose of the stem cells of the present invention can comprise, in various embodiments, about, at least, or no more than 3 million to 1 billion, 1×10⁵, 5×10⁵, 1×10⁶, 4×10⁶ 5×10⁶, 1×10⁷, 5×10⁷, 1×10⁸, 5×10⁸, 1×10⁹, 5×10⁹, 1×10¹⁰, 5×10¹⁰, 1×10¹¹ or more stem cells.

The pharmaceutical compositions provided herein can comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

Administration

Stem cells of the present invention can be administered to the patient suffering from a vascular-related degenerative disorder, e.g. multiple sclerosis, in the form of a pharmaceutical composition by any medically-acceptable route, e.g., a pharmaceutical composition suitable for intravenous, intra-arterial, intraperitoneal, intraventricular, intrasternal, intracranial, intramuscular, intrasynovial, intraocular, intravitreal, intracerebral, intracerebroventricular, intrathecal, intraosseous infusion, intravesical, transdermal, intracisternal, epidural, or subcutaneous administration. In specific embodiments, administration is by bolus injection or infusion directly into the site of the need for the cells. In a preferred embodiment of this aspect of the invention the cells are administered by intravenous and/orintrathecal administration. A preferred embodiment of this aspect of the invention contemplates administration through a balloon tipped catheter intravenously to the patient.

Another preferred embodiment of this aspect of the invention contemplates administration through a spinal needle intrathecally to the patient. In one embodiment, the stem cells are from a cell bank, e.g., a bone marrow stem cell bank. In another embodiment, a dose of stem cells is contained within a blood bag or similar bag, suitable for bolus injection or administration by catheter. Stem cells can be administered in a single dose, or in multiple doses. Where stem cells are administered in multiple doses, the doses can be part of a therapeutic regimen designed to relieve one or more symptoms of a vascular-related degenerative disorder, e.g., multiple sclerosis, or can be part of a long-term therapeutic regimen designed to prevent, or lessen the severity, of a chronic course of the disorder.

Dosage

A patient having, or experiencing, a symptom of a vascular-related degenerative disorder, e.g. multiple sclerosis, can be treated with a plurality of stem cells, at any time during the progression of the injury. For example, the patient can be treated immediately after showing symptoms of a vascular-related degenerative disorder, e.g. MS, or within 1, 2, 3, 4, 5, 6 days of the symptoms, or within 1, 2, 3, 4, 5, 6, 7, 8, 9, 10, 11, 12, 13, 13, 15, 16, 17, 18, 19, 20, 25, 30, 35, 40, 45, 50 days or more of the symptoms, or within 1, 2, 3, 4, 5, 6, 7, 8, 9, 10 or more years after the symptoms. The patient can be treated once, or multiple times during the clinical course of the injury. In a specific embodiment of this aspect of the present invention, said stem cells are administered to said patient within 21 days of development of one or more symptoms. In another specific embodiment of the method of treatment, said stem cells are administered to said patient within 14 days of development of one or more symptoms. In another specific embodiment of the method of treatment, said stem cells are administered to said patient within 7 days of development of one or more symptoms of a vascular-related degenerative disorder. In another specific embodiment of the method of treatment, said stem cells are administered to said patient within 48 hours of development of one or more symptoms. In another specific embodiment, said stem cells are administered to said patient within 24 hours of development of one or more symptoms. In another specific embodiment, said stem cells are administered to said patient within 12 hours of development of one or more symptoms. In another specific embodiment, said stem cells are administered to said patient within 3 hours of development of one or more symptoms.

In certain embodiments of the invention, the patient is an animal, more preferably a mammal. In even more preferred embodiments, the patient is a human patient. The patient can be a male or female subject. In certain embodiments, the subject is a non-human animal, such as, for instance, a cow, sheep, goat, horse, dog, cat, rabbit, rat or mouse.

Thus, in a specific embodiment of the method of treating MS provided herein, the therapeutically effective amount of stem cells is an amount sufficient to cause a decrease in the symptoms of MS as mentioned previously herein.

In one embodiment, the patient is administered a dose of about 200 million to 1 billion stem cells, preferably 300 million to 1 billion. Dosage, however, can vary according to the patient's physical characteristics, e.g., weight, and can range from 1 million to 1 billion autologous bone marrow stem cells per dose, preferably between 10 million and 1 billion per dose, including, or between 100 million and 500 million autologous bone marrow stem cells per dose, including 200 million, 300 million, 400 million, 500 million, 600 million, 700 million, 800 million or 900 million or in a preferred embodiment 400 million stem cells. The administration is preferably intravenous or intrathecal depending on the progression of the disease, but can be by any medically-acceptable route for the administration of live cells. In another embodiment cells to be used for patient doses can be frozen, e.g., cryopreserved for later use.

The actual number of cells or cell types administered however is meant to include any number of cells or cell types that are therapeutically effective in the treatment of multiple sclerosis. Administration of the cells can be all together in one dosage or may be administered separately or sequentially.

Without being limited to a particular theory, the vascular side of the blood brain barrier (BBB) has thus far been treated. In cases of serious or progressive vascular-related degenerative neurological disease, in particular MS, a second administration of a therapeutically effective amount of stem cells of the present invention which treat the CNS side of the BBB has been shown to further reduce the symptoms of MS. Progressive physical disability in MS is a result of a gradual yet continuous disease process within the brain and the spinal cord (CNS). Disease progression and the accompanying disability in MS are associated with axonal degeneration in addition to the hallmark demyelination. Direct delivery of the stem cells of the present invention into the cerebrospinal fluid (CSF) would therefore result in maximal proximity of stem cells to lesions without risk of damage to the CNS. Each of the initial open label clinical studies investigating the safety and feasibility of stem cells in MS used only the intrathecal route. (Karussis D, et al. 2010, Mohyeddin B M, et al, 2007. Yamout B, et al. 2010). Yet these studies have had limited success because they did not address the root cause of the disease which is the initial vascular injury that occurs within the veins, in particular within the cranial veins.

Restoration of neurological function in patients with disability may occur if reparative therapies can be developed specifically aimed at re-myelination and regeneration. Intrathecal (IT) therapy in this case is used to allow stem cells to access areas of the brain and the spinal cord, the CNS, through the CSF where demyelination has occurred. The initial trauma to the BBB in MS causes antigens, such as for example fibrinogen to cross the BBB and trigger a cascade of events. This includes release of pro-inflammatory cytokines and reactive oxygen species (ROS), which triggers an autoimmune response. The result is the formation of acute, inflammatory demyelinating lesions in the central nervous system or CNS.

The leakage of the antigen, fibrinogen, across the BBB, which is identified by (Davalos D, 2012), is inhibited by the first selective intravenous procedure of the present invention. Subsequently myelin repair or regeneration is essential for new nerve growth, allowing that the brain itself is plastic and capable of a certain degree of repair on its own, known as neuroplasticity. Stem cells delivered intravenously, even when selectively placed proximal to damaged venous structures are not able to easily cross into the CSF in enough numbers and therefore, have minimal therapeutic effect on myelin and neuronal tissues in the area of the CNS side of the BBB that have already been damaged. Infusing stem cells directly into the CSF allows these cells to reach the brain parenchyma and spinal cord where the actual lesion damage is present. Stem cells delivered by this method appear to repair previously damaged cellular structures (re-myelination), and restore function as evidenced by individuals receiving this protocol. To date many study participants who have been followed for 24 months have experienced recovery of function (partial or complete) and a significant reduction or absence of MS symptoms. It appears that normal regeneration and maintenance of tissue structures within the CNS is ongoing. Furthermore, since the process occurs naturally with the patient's own cells, there are no observed side-effects, unlike so many other types of medication and other chemotherapeutic regimes (IV and/or oral) used to treat the symptoms of MS.

Thus, in another aspect of the present invention there is provided an intrathecal administration of a therapeutically effective amount of the stem cells described herein subsequent to the intravenous administration of the therapeutically effective amount of the stem cells of the present invention to the internal jugular vein of the patient. In certain embodiments of this aspect of the invention the therapeutically effective amount of the stem cells are intrathecally administered within 1, 2, 3, 4, 5, 6, 7, 8, 9 10, 20, 30, 40, or 50 minutes of each other, or 1, 2, 3, 4, 5, 6, 7, 8, 9, 10, 12, 14, 16, 18, 20, or 22 hours of each other, or within 1, 2, 3, 4, 5, 6, 7, 8, 9, 10, 11, 12, 13, 14, 15, 16, 17, 18, 19 or 20 days or more of each other. Preferably, the intrathecal administration occurs 4 days after the intravenous administration. Preferably aspiration of CSF is performed in a procedure known as a spinal tap where the fluid was withdrawn through a needle via lumbar puncture. A spinal needle, a 22 or 26 gauge spinal needle, is stabilized in the bevel up position and inserted. An amount of CSF appropriate to the size and weight of the patient as determined by the neurologist is withdrawn, preferably between 80-150 mls into a series of 20 ml size syringes. The CSF was then refrigerated for preservation prior to its reinfusion in combination with the stem cells of the present invention during the intrathecal tissue transplant procedure. In an even more preferred embodiment of this aspect of the invention, the stem cells intrathecally administered are autologous bone marrow derived stem cells.

Another aspect of the present invention relates to methods of administration of the stem cells in accordance with the present invention in combination with a therapeutic agent. For example, the infusion of HMG-CoA reductase inhibitors with administration of stem cells according to the methods of the present invention enhances the efficacy (both growth and inhibition) of certain stem cells with corresponding receptors. These inhibitors also possess several other protective effects that might prove beneficial in the setting of allogeneic transplantation.

Any therapeutic agents or combination of therapeutic agents can be administered with the methods of the present invention. Therapeutic agents include for example Copaxone®, Aubagio®, Tysbari®, Tecfidera® and Lemtrada®. Such therapeutic agents can be administered in any combination with the stem cells of the present invention, at the same time or as a separate course of treatment. Additionally, the methods for treating a vascular-related degenerative disorder provided herein further encompass treating a vascular-related degenerative disorder by administering a therapeutically effective amount of stem cells in conjunction with one or more therapies or treatments used in the course of treating a vascular-related degenerative disorder, e.g. MS. The one or more additional therapies may be used prior to, concurrent with, or after administration of the stem cells. In some embodiments, the one or more additional therapies comprise surgical treatment.

The following examples are intended for illustration only and are not intended to limit the scope of the invention in any way.

### EXAMPLE

### Administration of Concentrated Autologous Bone Marrow Stem Cells to Multiple Sclerosis Patients

In this study 31 MS patients of varying MS disabilities based on the Kurtzke Disability Status Scale (EDSS), 3.0 to 6.5 EDSS received autologous bone marrow stem cells selectively transfused into their internal jugular vein concurrent to the expansion of a balloon tipped catheter to produce local retrograde flow as per the below. This therapeutic intervention is called selective stem cell placement (SSCP). The patients included had a progressive form of the disease and those who used walking assistive aides.

Methods

Aspiration of Bone Marrow

First, bone marrow was harvested from the iliac crest using a Rosenthal large bore 6.001×1.001×0.751 aspiration needle from Becton Dickinson. Local anesthetic was administered and aseptic technique was exercised. The skin was punctured with the bone marrow aspiration needle, with a stylet locked in place. Once the needle contacts the bone, it was advanced by slowly rotating the needle clockwise and counterclockwise until it entered the marrow cavity. Contact with the marrow cavity was usually noted by a sudden reduction in pressure. The depth of the penetration did not extend beyond an initial 1 cm. Once within the marrow cavity, the stylet was removed and the amount of bone marrow that was determined to be appropriate for the particular patient was aspirated (normally 150-200 mls). 20 ml syringes were used and bone marrow was EDTA-anti-coagulated at a recommended concentration of 1.50±0.25 mg/ml of bone marrow to preserve morphology. Harvested bone marrow aspirates were sent to the processing lab in the hospital where, after layering on density-gradient medium (Ficoll-Hypaque), it was washed using phosphate-buffered saline (1% DPBS), and re-suspended in heparinized normal saline (final volume: 5 mL). The aspirate was divided into two equal parts by volume in a biosafety cabinet (1300 Series Class II, Type A2 Biological Safety Cabinet (Fisher Scientific). The two aspirate volumes were transferred to Wintrobe tubes and centrifuged (Sorvall™ ST 16 Centrifuge Series) at 2800 RPM (@850 g) for 8 minutes to obtain a concentrated phase containing mononuclear cells. The plasma was separated (and discarded) from the mononuclear fraction in both volumes. Aliquots were taken from the above sample to measure total nucleated cell count, MNC count and viability testing using (Countess II Automated) cell-counter to assess viability of cell tissue. Final yield of all cell types for reinfusion was between 300 million-1 billion cells of all cell types present in bone marrow, and depended on the individual patient. The total yield was divided into two arrays of equal size by volume (Volume #1, and Volume #2) and the second volume was inoculated into appropriate culture media as specified by the microbiology laboratory and thereafter placed in a T Scientific Revco, UXF Series cryogenic storage unit (Fisher Scientific) using appropriate techniques known to microbiologists specific to keeping cells viable. Volume #2 stem cells were inoculated with DMSO and placed in a controlled-rate freezer (Front access CryoMed™ LN2, Thermo Fisher Scientific) to gradually reach a temperature of −80° C. before being transferred to the cryogenic storage unit (Revco™ ExF −86° C. Upright Ultra-Low Temperature Freezer). Before infusion, the frozen vial(s) was thawed in the water-bath to gradually reach 37° C. (Fisher Scientific™ Isotemp™ Digital-Control Water Baths: Model 205); centrifuged (Sorvall™ ST 16 Centrifuge Series) to separate the mononuclear fraction from the fluids and DMSO; extract the mononuclear fraction and discard the rest within a biological safety cabinet (1300 Series Class II, Type A2). To wash the cells, the extracted mononuclear fraction was mixed with saline and placed in the centrifuge once again, to separate the cell fraction. The saline and fluids were discarded and the washing process was repeated once again to get rid of any remaining traces of DMSO which are toxic to the stem cells at room temperature. The vial(s) containing the washed stem cells were stored in 2° to 8° C. (ice pack) in case the infusion was not immediate (delayed more than 15 minutes to an hour). The stem cells should be brought back to 37° C. by gently rubbing/rolling the vial, back and forth between two hands, before the infusion.

Balloon Venoplasty Technique

The patient was made ready for the second procedure in the Cathlab. The skin over the right groin was cleaned with Povidone Iodine and draped. In this procedure, catheterization was carried out via the right femoral vein with placement of a 5-F Check-Flo catheter (0.038 inch; 11-cm hemostatic introducer sheath). Once the skin and subcutaneous groin tissue were anesthetized by infiltration with about 5-10 cc Xylocaine 1% without adrenaline, the femoral vein was punctured with a small needle. A sheath was used to introduce catheters. Once the femoral vein was entered, a 180 cm Road Runner guide wire, was advanced through the needle into the vein. The needle was removed and a balloon catheter, with three lumens was introduced over the wire. The catheter was inserted into the vein and threaded upwards over the wire into the right atrium of the heart, and into the superior vena cava. From the superior vena cava, the physician accessed the Internal Jugular Veins. Lines and introducer sheaths were placed in the blood vessels with fluoroscopic guidance so that the Interventional Specialist ensured the expected venous trajectory is followed during insertion. The operator injected a radio-opaque contrast agent into the blood vessel through the catheter to verify venous obstructions and to observe blood flow characteristics in real-time via fluoroscopy.

The balloon, tightly wrapped around a catheter shaft to minimize its profile, was inserted through the skin at the femoral vein puncture site and into the narrowed section of the vessel. Diagnostic pancerebral angiography was performed with a Cobra Soft Radiopaque Tip Balloon Dilatation Catheter 5F 038 inch 120 cm vascular catheter. A compliant-type balloon catheter was employed because of their greater flexibility. The balloon was about 6 mm in width and 4 cm in length inflated to a maximal pressure of about 6 atm. The balloon was inflated with saline forced through a syringe which exerted pressure on a tortuous vein and caused blood to temporarily flow in an opposite direction, concomitant to the stem cell infusion. For retraction, a vacuum was pulled through the balloon to collapse it and the blood again flows normally. Inflations are maintained for about 30 to 60 seconds at a time and were repeated as necessary to infuse the entire volume of stem cell fluid mixture. If a lesion was present, the balloon maintains a fixed position over the lesion as the process is repeated.

Stem Cell Infusion via Catheter into Cervical and veins outside of the head, i.e. in the neck, or emissary veins.

Concurrent to the expansion of the balloon, a fluid mixture of stem cells in a 1-9 ratio of stem cells to normal saline was infused through the hollow multi-lumen catheter to the most distal points of the IJVs. Stem cells in the saline mixture were directed through the catheter in a direction opposite to the normal venous blood flow created by the expansion of the balloon and into the circulatory system of the affected venous tissue of the Cranial Vein at a point distal to the Sigmoid Sinus as confirmed by fluoroscopy. The fluid was held in the circulation for a short period and at a pressure which facilitates stem cell adherence to endoluminal walls, migration and absorption of the stem cells within the cranial venous system and/or the other biological functions of chemotaxis that allow cell differentiation, paracrine signaling cell recruitment, and penetration into the fibrinous adhesions to lay down basement tissue and matrix substances affected by the stem cells that form a permanent fibrous adhesion. The balloon was then deflated after the treatment period, and retro-infusion is terminated once all of the cell mixture has been infused. Immediately after the procedure, patients were taken to the Recovery Room where hospital staff monitors post-anesthesia and post-procedural care. Patients remained in recovery for about 1 hour while they were observed for any adverse events or pain. They were taken back to their hospital rooms once it was determined they were stable enough for discontinuance of acute care/observation. Post-operative management required patients to be kept under observation for two more days.

Lumbar Puncture Technique for Aspiration of Cerebrospinal Fluid (CSF)

Four 4 days after the venoplasty procedure, the more severely compromised patients, as determined pre-therapy on the Kurtzke Disability Status Scale, underwent a fourth procedure, aspiration of cerebrospinal fluid. Aspiration of CSF was performed in a procedure known as a spinal tap where the fluid was withdrawn through a needle via lumbar puncture. Wearing non-sterile gloves and with the patient on his or her side in the left lateral position, the L3-L4 interspace was located. That interspace and the one above and one below was palpated to find the widest space. The skin was cleaned with the anti-septic solution. A sterile drape was placed below the patient and a fenestrated drape was placed on the patient. The local anesthetic was administered. A skin wheal was raised and the deeper tissue anaesthetized. The needle was inserted all the way to the hub and drawn back to confirm that it is not in a blood vessel. This process described is continued, including the area above, below and very slightly to the sides. This process anaesthetizes the entire immediate area in the event that redirection of the spinal needle was required. Next, the spinal needle, which was a 22 or 26 gauge spinal needle, was stabilized in the bevel up position and inserted. The needle was positioned at a slightly cephalad angle, directing it toward the umbilicus. The needle was advanced slowly. Most times a characteristic pop was felt when the spinal canal was entered, and the stylet was withdrawn to observe the fluid flow. If no pop was felt at 4-5 cm, the stylet was withdrawn to observe the fluid flow from the needle. If no fluid was returned, the stylet was replaced, the needle was advanced or withdrawn a few millimeters, and a check was made for fluid return. This process was continued until fluid was successfully returned. Once the spinal canal was entered, the amount of CSF appropriate to the size and weight of the patient as determined by the neurologist was withdrawn, between 80-150 mls into a series of 20 ml size syringes. The CSF was then used for immediate reinfusion during the intrathecal tissue transplant procedure.

Subjects received transplants immediately post-CSF aspiration through the same spinal needle, post-intravenous procedure day 10. Local anesthesia and lumbar puncture (LP) was performed at the same L3-L4 interspace as previously described. The same 26-gauge spinal needle was fitted with a double lumen adaptor. One lumen was fitted with a 20 ml syringe and used for injection of 1×10⁶ autologous bone marrow derived stem cells. The other was fitted with a 20 lumen adaptor containing the CSF and both were concurrently infused with 400 μl CSF in equal measure. The cannula-mixed agents were slowly injected over a 2-3-minute period and the syringe was left in place for an additional minute to prevent leakage.

Results

Table 1. Study Subject Walk Test

The differences between pre and post-therapy walk times were analyzed. Despite wide ranging abilities, 27 of 31 subjects demonstrated changes of statistical significance. With an average of about 46 seconds to complete the pre-therapy walk test, the mean improvement post-therapy is 22.55%, adjusted from 24.97% (SD 15.7%, 95% Cl), significant to p<0.05. No adverse effects were observed. The results are shown in Table 1.

TABLE 1
Study Subject Walk Test

| | | | | | Type | | Yr | Yrs | Trial | Trial | | | Pre- | Post- | | |
|---|---|---|---|---|---|---|---|---|---|---|---|---|---|---|---|---|
| Treatment D | Pt # | Sex | Age | Walking | MS | EDSS | Dx | Dx | 1 | 2 | Diff | % | step | step | Diff | % |
| 2014 Oct. 15 | 16 | M | 62 | N | PPMS | 3.5 | 2013 | 1 | 8.70 | 8.42 | 0.28 | 0.03 | 12 | 12 | 0 | 0 |
| 2014 May 15 | 8 | F | 46 | N | SPMS | 4.0 | 2011 | 3 | 11.48 | 9.19 | 2.29 | 19.95 | 21 | 19 | 3 | 14 |
| 2014 Jun. 15 | 12 | F | 31 | N | PPMS | 4.0 | 2013 | 1 | 12.15 | 9.82 | 2.33 | 19.18 | 15 | 13 | 2 | 13 |
| 2014 Oct. 15 | 17 | F | 47 | N | RRMS | 4.0 | 2001 | 13 | 11.87 | 10.17 | 1.30 | 10.95 | 18 | 16 | 2 | 11 |
| 2014 Mar. 14 | 31 | M | 29 | N | PPMS | 4.0 | 2012 | 2 | 9.64 | 7.89 | 1.75 | 18.15 | 15 | 14 | 1 | 6 |
| 2014 Oct. 15 | 5 | F | 26 | N | RRMS | 4.5 | 2006 | 8 | 15.04 | 10.29 | 4.16 | 27.47 | 18 | 15 | 3 | 16 |
| 2013 Sep. 15 | 6 | F | 40 | N | SPMS | 4.5 | 1991 | 22 | 12.13 | 11.29 | 0.84 | 6.92 | 17 | 16 | 1 | 6 |
| 2014 Nov. 15 | 18 | F | 51 | WA | RRMS | 4.5 | 2003 | 11 | 11.90 | 10.97 | 0.93 | 7.82 | 22 | 17 | 5 | 23 |
| 2013 Sep. 15 | 23 | F | 57 | WA | PPMS | 4.5 | 1991 | 23 | 18.80 | 14.83 | 3.99 | 21.22 | 22 | 19 | 3 | 14 |
| 2014 Nov. 15 | 24 | F | 39 | N | PPMS | 5.0 | 2011 | 3 | 16.22 | 16.45 | 0.23 | 0.010 | 23 | 23 | 0 | 0 |
| 2012 Jul. 15 | 1 | M | 39 | N | PPMS | 5.5 | 2000 | 12 | 9.14 | 7.79 | 1.35 | 14.77 | 14 | 12 | 2 | 14 |
| 2014 Oct. 15 | 19 | F | 64 | N | SPMS | 5.5 | 1984 | 30 | 12.70 | 8.22 | 3.85 | 32.88 | 18 | 14 | 4 | 22 |
| 2014 Oct. 15 | 20 | F | 46 | N | RRMS | 5.5 | 2007 | 6 | 10.27 | 9.11 | 1.53 | 14.04 | 18 | 14 | 4 | 22 |
| 2012 Sep. 15 | 2 | F | 52 | WA | RRMS | 6.0 | 1995 | 17 | 128.35 | 99.3 | 29.02 | 22.61 | 31 | 26 | 7 | 23 |
| 2014 Mar. 14 | 9 | M | 59 | WA | SPMS | 6.0 | 2008 | 6 | 28.15 | 25.18 | 2.97 | 13.81 | 20 | 18 | 2 | 10 |
| 2014 Mar. 15 | 10 | F | 58 | WA | SPMS | 6.0 | 1995 | 18 | 41.50 | 31.57 | 9.93 | 23.93 | 15 | 10 | 5 | 33 |
| 2014 Mar. 15 | 11 | F | 63 | WA | SPMS | 6.0 | 1998 | 16 | 28.06 | 19.60 | 8.46 | 30.14 | 22 | 17 | 5 | 23 |
| 2015 Feb. 15 | 26 | M | 57 | N | PPMS | 6.0 | 2011 | 3 | 15.01 | 8.00 | 7.01 | 46.70 | 20 | 10 | 10 | 50 |
| 2015 Jan. 15 | 27 | F | 27 | WA | SPMS | 6.0 | 2013 | 1 | 32.03 | 23.97 | 8.06 | 25.16 | 29 | 23 | 6 | 21 |
| 2015 Jan. 15 | 29 | F | 55 | WA | RRMS | 6.0 | 1984 | 25 | 24.57 | 20.00 | 4.57 | 18.60 | 25 | 20 | 5 | 20 |
| 2013 Jan. 20 | 3 | F | 47 | WA | PPMS | 6.5 | 1998 | 15 | 138.77 | 124.48 | 14.29 | 10.30 | 39 | 37 | 2 | 5 |
| 2014 Jun. 15 | 4 | F | 58 | WA | RRMS | 6.5 | 2005 | 9 | 74.93 | 56.37 | 18.56 | 24.77 | 24 | 21 | 3 | 13 |
| 2014 Jul. 15 | 7 | F | 52 | WA | PPMS | 6.5 | 1997 | 17 | 32.78 | 22.00 | 10.78 | 32.89 | 28 | 23 | 5 | 18 |
| 2014 Oct. 15 | 13 | F | 46 | WA | SPMS | 6.5 | 1994 | 20 | 141.70 | 101.63 | 40.07 | 28.28 | 33 | 28 | 5 | 15 |
| 2014 Nov. 15 | 14 | F | 44 | WA | PPMS | 6.5 | 2012 | 2 | 145.72 | 98.37 | 47.35 | 32.49 | 30 | 25 | 5 | 17 |
| 2015 Jan. 15 | 15 | F | 46 | WA | PPMS | 6.5 | 2003 | 11 | 45.26 | 26.29 | 18.95 | 41.71 | 30 | 21 | 9 | 30 |
| 2014 Jul. 15 | 21 | F | 60 | WA | PPMS | 6.5 | 1993 | 21 | 28.72 | 22.20 | 6.52 | 22.70 | 22 | 19 | 3 | 14 |
| 2013 Feb. 20 | 22 | M | 42 | WA | SPMS | 6.5 | 2007 | 6 | 33.00 | 34.51 | 0.46 | 0.001 | 17 | 17 | 0 | 0 |
| 2013 Sep. 20 | 25 | F | 46 | WA | SPMS | 6.5 | 1994 | 20 | 136.60 | 97.12 | 39.5 | 28.90 | 32 | 27 | 5 | 15 |
| 2014 Oct. 15 | 28 | M | 51 | WA | PPMS | 6.5 | 2010 | 4 | 12.03 | 9.15 | 2.60 | 21.49 | 16 | 12 | 4 | 25 |
| 2013 Feb. 10 | 30 | F | 49 | N | SPMS | 7.0 | 2003 | 11 | 39.28 | 7.39 | 31.89 | 81.19 | 30 | 13 | 17 | 94 |
| Mean | | | 50 | | | | | 12 | 45.95 | 31.02 | 11.64 | 24.97 | | | 4.6 | 21.0 |

WA—Walking with assistance
W—Walking without assistance

As can be seen from the results shown in table the method of the present invention significantly improved the time taken for a patient to complete the Timed 25 Foot Walk post-therapy regardless of degree of disability.

The methods and compositions disclosed herein are not to be limited in scope by the specific embodiments described herein. Indeed, various modifications of the methods and compositions in addition to those described will become apparent to those skilled in the art from the foregoing description and accompanying figures. Such modifications are intended to fall within the scope of the appended claims. Various publications, patents and patent applications are cited herein, the disclosures of which are incorporated by reference in their entireties.

### REFERENCES

1. Alharbi F M (2015) Update in vitamin D and multiple sclerosis. Neurosciences. 2015; 20(4): 329-335 doi: 10.17712/nsj.2015.4.20150357
2. About M S: What causes MS? [homepage on internet] National Multiple Sclerosis Society; [cited 2013 Mar. 4] Available from: http://www.nationalmssociety.org/about-multiple-sclerosis/what-we-know-about-ms/what-causes-ms/index.aspx.
3. Bain B (1995) The Bone Marrow Aspirate of Healthy Subjects. Br J Hematol. 1996;(94):206-209.
8. Banks W A et al (2013) The blood-brain barrier and immune function and dysfunction. Neurobiology of Disease. 2013; 37:26-32.
9. Beck A T et al (1988) Psychometric properties of the Beck Depression Inventory: Twenty-five years of evaluation. Clinical Psychology Review. 1988; 8(1): 77-100.
10. Behrman A et al (2006) Neuroplasticity After Spinal Cord Injury and Training: An Emerging Paradigm Shift. Rehabilitation and Walking recovery. Phys Therapy. October 2006; 86 (10): 10.2522/ptj. 20050212.
11. Bergers G et al (2005) The role of the pericytes in blood-vessel formation and maintenance. Neuro-Oncol. October 2005; 7(4):452-464.
12. Brazzini A. (2010) Intraarterial autologous implantation of adult stem cells for patients with Parkinson's disease. J Vasc Intery Radiol. 2010; 21:443-451.
13. Chaudhuri A et al (2004) Multiple Sclerosis Is Not An Autoimmune Disease. Arch Neurol. October 2004; 61:1610-1612.
14. Civin C I et al (1984) Antigenic analysis of hematopoiesis. III. A hematopoietic progenitor cell surface antigen defined by a monoclonal antibody raised against KG-1a cells. J Immunol. July, 1984; 133(1):157-165.
15. Cohen S R et al (1980) A diagnostic index of active demyelination: Myelin basic protein in cerebrospinal fluid. Ann of Neurol. July. 1980; 8(1):25-31.
16. Correale J et al (2006) The blood-brain barrier in multiple sclerosis: Functional roles and therapeutic targeting. Autoimmunity. 2006; 40:148-160.
17. Davalos D et al (2012) Fibrinogen-induced perivascular microglial clustering is required for the development of axonal damage in neuroinflammation. Nat Commun. 2012; 3:1227 doi: 10.1038/ncomms2230.
18. De Vries H et al (1997) The Blood-Brain Barrier in neuroinflammatory diseases. Pharmacological Reviews. 1997; 49: 143-156.
19. D'haeseleer M et al (2011) Vascular aspects of multiple sclerosis. Lancet Neural. July.2011; (10):657-666.
20. Dimmeler S et al (2001) HMG-CoA reductase inhibitors (Statins) increase endothelial progenitor cells via the PI 3-kinase/Akt Pathway. J Clin Invest. 2001;108:391-397.
21. Fischer U M et al (2009) Pulmonary Passage is a Major Obstacle for I.V. Stem Cell Delivery: The Pulmonary First Pass Effect. Stem Cells and Development. 2009; 18(5): 683-601.
22. Francis M P et al (2010) Isolating adipose-derived mesenchymal stem cells from lipoaspirate blood and saline fraction. Organogenesis. 2010. 6:1, 11-14, DOI: 10.4161/org.6.1.10019
23. Furlani D et al (2009) Is the intravascular administration of stem cells safe? Mesenchymal stem cells and intravital microscopy. Microvascular Research. May.2009; (77): 370-376.
24. Freire M A (2012) Pathophysiology of neurodegeneration following traumatic brain injury. West Indian Med J. October 2012; 61(7): 751-755.
25. Ge Y et al (2008) 7T MRI: New Vision of Microvascular Abnormalities in Multiple Sclerosis. Arch Neurol. June 2008; 65(6): 812-816.
26. Goldberg A et al (2011) Measurement error and minimum detectable change in 4-meter gait speed in older adults. Aging Clin Exp Res. 2011; 23(5-6):406-412.
27. Gulcher J R et al (2004) Is multiple sclerosis an autoimmune disease? Clin Neurosci. 1994; 2(3-4): 246-252.
28. Hartung D M et al (2015) The cost of multiple sclerosis drugs in the US and the pharmaceutical industry. Too big to fail. Neurology. May.2015; 84(21): 2185-2192.
29. Hobart J et al (2013) Timed 25-foot walk: direct evidence that improving 20% or greater is clinically meaningful in MS. Neurology. April 2013; 80(16): 1509-1517.
30. IUIS-WHO Nomenclature Subcommittee (1984) Nomenclature for clusters of differentiation of antigens defined on human leukocyte populations.
31. Karussis D et al (2010) Safety and immunological effects of mesenchymal stem cell transplantation in patients with multiple sclerosis and amyotrophic lateral sclerosis. Arch Neurol. October 2010; 67(10): 1187-1194.
32. Kaufman M et al (2000) The significant change for the Timed 25-Foot Walk in the multiple sclerosis functional composite. Mult Scler 2000; 6: 286-290.
33. Kaufman M et al (2013) Clinically meaningful performance benchmarks in MS. Timed 25-Foot Walk. Neurology. November 2013; 81(21):1856-1863.
34. Kieseier B et al (2012) Assessing walking disability in multiple sclerosis. Mult Scler 2012; 18:914-924.
35. Kirk J et al (2003) Tight junctional abnormality in multiple sclerosis white matter affects all calibres of vessels and is associated with blood-brain barrier leakage and active demyelination. The Journal of Pathology. 2003; 201(2): 319-327.
36. Kirouac D C et al (2008) The Systematic Production of cells for Cell Therapies. Cell Stem Cell. October 2008; DOI 10.1016/j.stem.2008.09.0
37. Kresbach P H et al (1999) Bone Marrow Stromal cells: Characterization and Clinical Application. Crit Rev Oral Biol Med. 1999; 12(2):165-181.
38. Krizanac-Bengez I et al (2004) The cerebral vasculature as a therapeutic target for neurological disorders and the role of shear stress in vascular homeostasis and pathophysiology. Neurological Research. 2004; 26(8): 846-853.
39. Kumar D et al (2011) Jean-Martin Charcot: The Father of Neurology. Clin Med Rev. March 2011; 9(1):46-49.
40. Kurtzke J F (1994) Clinical definition for multiple sclerosis treatment trials. Ann Neurol. 1994; 36 Suppl:S73-9.
41. Lamagna C (2006) The bone marrow constitutes a reservoir of pericyte progenitors. Journal of Leukocyte Biology. October 2006; 80(4): 677-681.
42. Larochelle C et al (2011) How do immune cells overcome the blood-brain barrier in multiple sclerosis? FEBS Letters, 585, doi:10.1016/j.febslet.2011.04.066
43. Lodi D et al (2011) Stem cells in clinical practice: applications and warnings. J Exp Clin Cancer Res. 2011; 30(1): 9 Published online 2011 Jan. 17 doi: 10.1186/1756-9966-30-9
44. Lodish H et al (2000) Molecular Cell Biology. 4th edition. New York: W. H. Freeman; 2000. Available from: https://www.ncbi.nlm.nih.gov/books/NBK21475/45.
45. Lublin F D et al (2014) “Defining the clinical course of multiple sclerosis. The 2013 revisions”. Neurology. July 2014; 83(3): 278-286.
46. McCuigan C et al (2004) The multiple sclerosis impact scale (MSIS-29) is a reliable and sensitive measure. J Neurol Neurosurg Psychiatry. February 3004; 75(2): 266-269.
47. McCuigan C et al (2004) Confirming the validity and responsiveness of the Multiple Sclerosis Walking Scale-12 (MSMW-12). Neurology. 2004; 62:2103-2105.
48. Middleton A et al (2015) Walking Speed: The Functional Vital Sign. J Aging Phys Act. April 2015; 23(2): 314-322.
49. Miller C J et al (2013) The Interplay between cell signaling and mechanics in developmental processes. Nature Reviews Genetics. 2013; 14:733-744.
50. Minangar A et al (2003) Blood-brain barrier disruption in multiple sclerosis. Mult Scler. 2003: 6: 540-549.
51. Mohyeddin B M et al (2007) Does mesenchymal stem cell therapy help multiple sclerosis patients? Report of a pilot study. Iran J Immunol. March 2007; 4(1): 50-57.
52. Nordqvist, C., Multiple Sclerosis: Causes, Symptoms and Treatments (2016, March 9). Retrieved from http://www.medicalnewstoday.com/articles/37556.php?page=2.
53. http://www.medicalnewstoday.com/articles/37556.php
54. Oswald J et al (2004) Mesenchymal Stem Cells Can Be Differentiated into Endothelial Cells In Vitro. Stem Cells. 2004; 22:377-384.
55. Ozerdem U et al (2005) Contribution of bone marrow-derived pericyte precursor cells to corneal vasculogenesis. Invest Opthalmol Vis Sci. October 2005; 46(10): 3502-3506.
56. Passweg J R et al (2012) Hematopoietic stem cell transplantation: a review and recommendations for follow-up care for the general practitioner. Swiss Med Weekly. October 2012; 142: W1396.
57. Paty D W et al (1993) Interferon beta-lb is effective in relapsing-remitting multiple sclerosis. 11. MRI analysis results of a multicenter, randomized, double-blind, placebo-controlled trial. Neurology. April 1993; 43:662-667
58. Peerani R et al (2010) Enabling stem cell therapies through synthetic stem cell-niche engineering. J Clin Invest. January 2010; 120(1): 60-70.
59. Pietrangelo A et al (2015) Multiple Sclerosis by the Numbers: Facts, Statistics, and You. March 2015; Retrieved from Healthline. http://www.healthline.com/health/multiple-sclerosis/facts-statistics-infographic
60. Polliack M L et al (2001) Late-onset multiple sclerosis. J Am Geriatr Soc. February 2001; 49(2):168-171
61. Polman C et al (2011) Diagnostic criteria for Multiple Sclerosis: 2010 Revisions to the McDonald Criteria. Ann Neurol. 2011; (69):292-302.
62. Poser C M et al (1983) New diagnostic criteria for multiple sclerosis: Guidelines for research protocols. Ann Neurol. March 1983; 13(3):227-231.
63. Puthoff M L et al (2013) Reliability and responsiveness of gait speed, five times sitto stand, and hand grip strength for patients in cardiac rehabilitation. Cardiopulm Phys Ther J. 2013; 24(1):31-37.
64. Rice C M (2014) Disease Modification in multiple sclerosis: an update. Prac Neuro. 2014; (14):6-13. doi:10.1136/practneurol-2013-000601
65. Schelling F (1986) Damaging venous reflux into skull and spine. Relevance to MS. Medical Hypotheses October 1986; 21(2):141-148.
66. Schelling F (2012) Chronic cerebrospinal venous insufficiency in multiple sclerosis: Weighing the findings. Sang Thrombose Vaisseaux. 2012; 24(9):394-404
67. Schwid SR et al (2002) Quantitative functional measures in MS: what is a reliable change? Neurology. 2002; 58:1294-1296.
68. Shi S et al (2003) Perivascular Niche of Postnatal Mesenchymal Stem Cells in Human Bone marrow and Dental Pulp. Journal of Bone and Mineral Research. 2003; 18(4):606=704.
69. Simpson Jr. S et al (2011) Latitude is significantly associated with the prevalence of multiple sclerosis: a meta-analysis. J Neurol Neurosurg Psychiatry (2011); 82: 1132-1141.
70. Sipe J et al (1984) A neurologic rating system (NRS) for use in multiple sclerosis. Neurology. October 1984; 34(10): 1368.
71. Sobel R A et al (1984) The immunopathology of experimental allergic encephalomyelitis. II. Endothelial cell Ia increases prior to inflammatory cell infiltration. J. Immunol. May. 1984; 132(5):2402-2407.
72. Song S et al (2005) PDGFR-β perivascular progenitor cells in tumors regulate pericyte differentiation and vascular survival. Nature Cell Biology. 2005; 7:870-879. Published online: 21 Aug. 2005 | doi:10.1038/ncb1288
73. Sotiropoulos P A et al (2006) Characterization of the Optimal Culture Conditions for Clinical Scale Production of Human Mesenchymal Stem Cells. Stem cells. 2006; 24:462-471.
74. Srivastava A (2010) Multiple Injections of in vitro expanded autologous bone marrow stem cells for cervical level spinal cord injury—a case report. J Stem Cells Regen Med. October 2010; 6(3): 175-176.
75. Steinman L. (2001) Multiple sclerosis: a two-stage disease. Nature Immunology. 2001; 2: 762-764.
76. Traugott U (1989) Detailed analysis of early immunopatholgic events during lesion formation in acute experimental autoimmune encephalomyelitis. Cell Immunol. March 1989; 119(1):114-129.
77. Tsivgoulis G et al (2015) “Liberation treatment” for chronic cerebrospinal venous insufficiency in multiple sclerosis: the truth will set you free. Brain Behay. January 2015; 5(1): 3-12.
78. Tumani H (2008) Corticosteroids and plasma exchange in multiple sclerosis. Neurol. December 2008; 255(Suppl 6): 36-42. doi: 10.1007/s00415-008-6007-9.
79. Tuohy et al (1988) A synthetic peptide trom myelin proteolipid protein induces experimental allergic encephalomyelitis. J. Immunol. August 1988; 141(4): 1126-1130
80. IUIS-WHO Nomenclature Subcommittee. Nomenclature for clusters of differentiation (CD) of antigens defined on human leukocyte populations. Bulletin of the World Health Organization. 1984; 62(5):809-815.
81. Van Der Sanden Bet al (2010) Optimizing stem cell culture. J Cell Biochem. November 2010; 111(4): 801-807.
82. Vidyasagar D D (2012) Index of CD34+ Cells and Mononuclear Cells in the Bone Marrow of Spinal Cord Injury Patients of Different Age Groups: A Comparative Analysis. Bone Marrow Research. Volume 2012, ArticleID 787414, 8 pages doi: 10.1155/2012/787414
83. Voog J et al (2010) Stem Cells and the Niche: A Dynamic Duo. Cell Stem Cell. February 2010; 6(2): 103-115.
84. Wagey R et al (2014) Mesenchymal Stem and Progenitor Cells: Problems, Potential and Promise. J Stem Cells Res, Rev & Rep. 2014; 1(4): 1016. ISSN: 2381-9073
85. Watt S et al (2013) The angiogenic properties of mesenchymal stem/stromal cells and their therapeutic potential. British Medical Bulletin. 2013; 108:25-53. DOI:10.1093/bmb/ldt031
86. Weinshenker B G (1994) Natural History of Multiple Sclerosis. Ann Neurol. 1994; 36: S6-S11
87. Wootla B et al (2012) Is Multiple Sclerosis an Autoimmune Disease? Autoimmune Diseases. Volume 2012, Article ID 969657, 12 pages doi:10.1155/2012/969657
88. Yamanaka S (2012) Induced Pluripotent Stem Cells: Past, present and future. Cell Stem Cell. June. 2012; 10(6):678-684.
89. Yamout B et al (2010) Bone marrow mesenchymal stem cells transplantation in patients with multiple sclerosis: a pilot study. J Neuroimmunol. October 2010; 227(1-2): 185-189.
90. Yu J et al (2003) Pluripotent stem cell lines. Genes Dev. August 2008: 22(15): 1987-1997.
91. Zlokovic B (2008) The Blood-Brain Barrier in Health and Chronic Neurodegenerative Disorders. Neuron. January 2008; DOI 10.1016/j.neuron.2008.01.003

What is claimed is:

1. A method of treating a patient having a vascular-related degenerative neurological disease comprising intravenously administering to the patient a therapeutically effective amount of stem cells to the patient.

2. The method of claim 1 wherein the vascular-related degenerative neurological disease is selected from the group consisting of optic neuritis, Devic's disease, Lyme's disease, migraine headache, cluster headache, vascular dementia, ischemic depression, ischemic stroke, motor neuron disease and multiple sclerosis.

3. The method of claim 1 wherein the vascular-related degenerative neurological disease is multiple sclerosis.

4. The method of claim 3 wherein the intravenous administration is to a vein of the patient wherein the vein is selected from the group consisting of an internal jugular vein, azygous vein and vertebral vein.

5. The method of claim 4 wherein the intravenous administration is to an internal jugular vein of the patient.

6. The method of claim 5 wherein the stem cells are autologous bone marrow stem cells.

7. The method of claim 5 wherein the autologous bone marrow stem cells are intravenously administered into the internal jugular veins of the patient whereby the cells migrate to the cranial veins of the brain of the patient.

8. The method of claim 7 wherein the cells are administered to and maintained in the cranial veins by expansion of a device wherein the expansion of the device occludes the vein proximal to the point of release of the stem cells.

9. The method of claim 8 wherein after administration of the cells a local retrograde flow of the cells is maintained in the cranial veins.

10. The method of claim 9 wherein the device is a balloon-tipped catheter with the cells releasably associated therewith.

11. The method of claim 6 wherein the therapeutically effective amount of the autologous bone marrow stem cells comprises between about 300 million and about 500 million autologous bone marrow stem cells isolated from the patient.

12. The method of claim 11 wherein the cells are intravenously administered to the internal jugular vein of the patient for a period of time not less than about 30 seconds to about not more than about 60 seconds.

13. A pharmaceutical composition comprising the cells of claim 11 and a pharmaceutically acceptable medium.

14. A pharmaceutically acceptable medium for transferring autologous bone marrow stem cells comprising Dulbecco's-modified Eagle's Medium-Low Glucose, GlutaMAX™, penicillin, streptomycin and fetal calf serum.

15. A method of treating a patient having a vascular-related degenerative neurological disease comprising the steps of:
selectively transfusing a therapeutically effective amount of autologous bone marrow stem cells into an internal jugular vein of the patient; and
occluding the jugular vein proximally to the point of insertion of the cells through expansion of a balloon tipped catheter thereby maintaining a local retrograde flow of the cells in the cranial vein.

16. The method of claim 15 wherein the vascular-related degenerative neurological disease is multiple sclerosis.

17. The method of claim 15 comprising the further step of intrathecally administering a therapeutically effective amount of autologous bone marrow stem cells to the patient's spinal canal subsequent to the administration of the cells to an internal jugular vein of the patient.

18. The method of claim 17 wherein the vascular-related degenerative neurological disease is multiple sclerosis.

19. The method of claim 18 wherein the intravenous administration is into an internal jugular vein using a balloon tipped catheter which expands proximally to the cells to occlude the jugular vein such that the cells are maintained in the cranial veins and undergo a local retrograde flow and the intrathecal administration is to the patient's spinal canal by lumbar puncture whereby the intrathecal administration is performed about 4 days subsequent to the intravenous administration.

20. A method of preparing a pharmaceutical composition comprising a therapeutically effective amount of autologous stem cells and a pharmaceutically acceptable carrier comprising removing the cells from the patient to be treated; isolating the cells and mixing the cells with the medium of claim 14.

22. A device comprising autologous bone marrow stem cells releasably associated therewith.

23. The device of claim 22 wherein the device is an expandable balloon tipped catheter.
